(19) United States
(12) Patent Application Publication
(10) Pub. No.: US 2018/0080021 A1
(43) Pub. Date: Mar. 22, 2018

(54) SIMULTANEOUS SEQUENCING OF RNA AND DNA FROM THE SAME SAMPLE

(71) Applicant: The Board of Trustees of the Leland Stanford Junior University, Stanford, CA (US)

(72) Inventors: Jason A. Reuter, Palo Alto, CA (US); Michael Snyder, Stanford, CA (US); Damek V. Spacek, Palo Alto, CA (US)

(21) Appl. No.: 15/704,159

(22) Filed: Sep. 14, 2017

Related U.S. Application Data
(60) Provisional application No. 62/396,155, filed on Sep. 17, 2016.

(51) Int. Cl. C12N 15/10; C12Q 1/68

(52) U.S. Cl. CPC C12N 15/1082; C12N 9/22; C12N 15/1096; C12Q 1/6827

(57) ABSTRACT

Methods, compositions, and kits for simultaneously generating RNA and DNA sequencing libraries are disclosed. In particular, the disclosed methods allow high-throughput amplification and sequencing of both RNA and DNA from a single sample source without the need to divide the sample to separate nucleic acids from each other. All of the preparation steps from harvesting nucleic acids from cells or tissue to preparing RNA and DNA sequencing libraries can be performed with the RNA and DNA pooled together. This technology streamlines generation of DNA and RNA sequencing data in combination and makes possible comprehensive genomic and transcriptomic profiling of a cell population concurrently.

### CROSS-REFERENCE TO RELATED APPLICATION

This application claims benefit under 35 U.S.C. § 119(e) of provisional application 62/396,155, filed Sep. 17, 2016, which application is hereby incorporated by reference in its entirety.

### STATEMENT REGARDING FEDERALLY SPONSORED RESEARCH OR DEVELOPMENT

This invention was made with government support under contracts HG000044 and HG007735 awarded by the National Institutes of Health. The government has certain rights in the invention.

### TECHNICAL FIELD

The present invention pertains generally to nucleic acid sequencing technology. In particular, the invention relates to a method of simultaneously generating RNA and DNA sequencing libraries from the same sample.

### BACKGROUND

Integration of both DNA and RNA sequencing data enables a variety of analyses that are useful for exploring the genetics of normal phenotypic variation and disease. In addition to enumerating global patterns of gene expression, RNA sequencing data provides an orthogonal verification of DNA variant calls and can be used to prioritize expressed candidates, which are more likely to exert biologic effects. In cancer for example, roughly a third of the somatic single nucleotide variants (SNVs) that fall within coding regions can also be observed in the RNA (Shah et al. (2012) Nature 486(7403):395-299), providing a biologic filter for candidate driver mutations. Furthermore, combined DNA and RNA profiling is useful for characterizing regulatory variation (Grubert et al. (2015) Cell 162:1051-1065, Stranger et al. (2007) Nat. Genet 39:1217-1224, Ongen et al. (2014) Nature 512(7512):87-90), RNA-editing (Li et al. (2009) Science 324(5931):1210-1213) and allele-specific expression (Tuch et al. (2010) PLoS One 5(2):e9317, Su et al. (2004) Proc. Natl. Acad. Sci. U.S.A. 101:6062-6067, Lappalainen et al. (2013) Nature 501:506-511), important contributors to phenotypic diversity and disease.

Currently, most integrative experiments are performed in parallel and on distinct cell populations, a strategy that requires lengthy library preparation times and potentially exacerbates variability due to sample heterogeneity. Single cell integrative sequencing approaches, Genome and Transcriptome-sequencing (G&T-seq, Macaulay et al. (2015) Nat. Methods 12:519-522) and gDNA and mRNA sequencing (DR-seq, Dey et al. (2015) Nat. Biotechnol. 33(3):285-289, Supplementary Information), have recently produced the first genome-wide glimpses of the correlation between copy number and expression at a cellular level. However, due to the large technical variance and coverage gaps inherent in current single cell sequencing approaches, these new methods have limited utility in contexts where more comprehensive genomes and transcriptomes are required. Moreover, both methods still require the DNA and RNA libraries to be generated independently.

Thus, there remains a need for improved methods of generating DNA and RNA sequence data in combination that would allow comprehensive genomic and transcriptomic analysis of a sample.

### SUMMARY

The present invention relates to a method for simultaneously generating RNA and DNA sequencing libraries from the same sample.

In one aspect, the invention includes a method of sequencing RNA and DNA, the method comprising: a) providing a biological sample comprising nucleic acids; b) isolating the nucleic acids from the sample; c) fragmenting the DNA by contacting the nucleic acids with a Class 2 transposase loaded with a transposable oligonucleotide adapter comprising a common priming site for DNA-specific amplification, wherein the transposase catalyzes cleavage of the DNA into a plurality of DNA fragments and ligation of the oligonucleotide adapter to each DNA fragment at the 5′ ends of its DNA strands; d) fragmenting the RNA by contacting the nucleic acids with RNase III, such that the RNase III cleaves the RNA into a plurality of RNA fragments; e) ligating a 3′ oligonucleotide adapter comprising a 3′ common priming site for RNA-specific amplification to the 3′ end of each RNA fragment using an RNA ligase; f) ligating a 5′ oligonucleotide adapter comprising a 5′ common priming site for RNA-specific amplification to the 5′ end of each RNA fragment using an RNA ligase; g) adding reverse transcriptase such that a plurality of cDNA fragments is produced from the plurality of RNA fragments; h) amplifying the plurality of DNA fragments with a set of DNA indexing primers that selectively bind to the common priming sites for DNA-specific amplification to produce a plurality of DNA amplicons, wherein each amplicon comprises a common priming site for DNA-specific sequencing and a DNA index sequence; i) amplifying the plurality of cDNA fragments with a set of RNA indexing primers that selectively bind to the common priming sites for RNA-specific amplification to produce a plurality of cDNA amplicons, wherein each amplicon comprises a common priming site for RNA-specific sequencing and an RNA index sequence; and j) sequencing the DNA amplicons and the cDNA amplicons, wherein the DNA index sequence is used to identify DNA sequences and the RNA index sequence is used to identify RNA sequences. All steps of the method may be performed with the RNA and DNA nucleic acids pooled together. In one embodiment, steps (a)-(j) are carried out in one container. Fragmentation of the DNA and RNA may be performed simultaneously. The transposase used in the practice of the invention is typically a hyperactive transposase. For example, the transposase may be a Tn5 transposase or hyperactive Tn5 transposase variant.

Nucleic acids may be obtained from any source. Sources of nucleic acid molecules include, but are not limited to, organelles, cells, tissues, organs, and organisms. In certain embodiments, the DNA and the RNA are from a single cell or a selected population of cells of interest. The cell may be a live cell or a fixed cell. In certain embodiments, the DNA and RNA are obtained from a biological sample comprising a eukaryotic cell (e.g., animal, plant, fungus, or protist), prokaryotic cell (e.g., bacterium or archaeon), or a virus. For example, the biological sample may comprise a genetically aberrant cell, cancer cell, or rare blood cell. In one embodiment, the cell is a human cell. In another embodiment, the DNA and the RNA are obtained from a sample of micro-dissected tissue or a biopsy.

In certain embodiments, isolating the nucleic acids comprises lysing a cell in the biological sample and extracting the nucleic acids from the cell.

In another embodiment, the method further comprises removing ribosomal RNA from the nucleic acids prior to fragmenting the DNA and RNA.

In certain embodiments, amplification of the DNA and cDNA fragments is performed using digital droplet PCR or clonal amplification (e.g., emulsion PCR or bridge amplification).

In another embodiment, at least one RNA indexing PCR primer comprises a nucleotide sequence of SEQ ID NO:1 or a nucleotide sequence having at least 95% identity to the nucleotide sequence of SEQ ID NO:1.

In another embodiment, the method further comprises purifying the RNA or DNA prior to amplification or sequencing. For example, DNA or RNA fragments may be purified by immobilization on a solid support such as, but not limited to, adsorbent beads, magnetic beads, or silica, or by gel filtration, reverse phase, ion exchange, or affinity chromatography. Alternatively, an electric field-based method can be used to separate DNA or RNA fragments from one another and other molecules. Exemplary electric field-based methods include polyacrylamide gel electrophoresis, agarose gel electrophoresis, capillary electrophoresis, and pulsed field electrophoresis.

Sequencing of DNA or cDNA may comprise paired-end sequencing or single-read sequencing. In certain embodiments, sequencing comprises performing a next-generation sequencing (NGS) technique. In another embodiment, the method further comprises assembling DNA sequences or cDNA sequences to produce longer sequences. In certain embodiments, overlapping sequence reads are assembled into contigs or full or partial contiguous sequences of a nucleic acid of interest (e.g. DNA gene, intergenic regulatory region, or RNA coding or non-coding transcript).

In another embodiment, the method further comprises identifying a mutation in the DNA or RNA. A mutation may comprise, for example, an insertion, a deletion, or a substitution. For example, a mutation may include a single nucleotide variation, gene fusion, translocation, inversion, duplication, frameshift, missense, nonsense, or other mutation associated with a phenotype or disease of interest.

In another embodiment, the method further comprises detecting genomic copy number variation.

In another embodiment, the method further comprises performing transcriptome quantification or isoform analysis.

In another aspect, the invention includes a kit for simultaneously generating RNA and DNA sequencing libraries as described herein. The kit may include a Class 2 transposase; a transposable oligonucleotide comprising an oligonucleotide adapter comprising a common priming site for DNA-specific amplification; a 5′ oligonucleotide adapter comprising a 5′ common priming site for RNA-specific amplification; a 3′ oligonucleotide adapter comprising a 3′ common priming site for RNA-specific amplification; an RNase III; an RNA ligase; a reverse transcriptase; a DNA polymerase; a set of DNA indexing PCR primers; and a set of RNA indexing PCR primers. The kit may further comprise information, in electronic or paper form, comprising instructions for performing the methods described herein for simultaneously generating RNA and DNA sequencing libraries. In certain embodiments, the kit may further comprise reagents for performing clonal amplification, digital droplet PCR, or next-generation sequencing. In another embodiment, the kit comprises a set of RNA indexing PCR primers comprising a primer comprising a nucleotide sequence of SEQ ID NO:1 or a nucleotide sequence having at least 95% identity to the nucleotide sequence of SEQ ID NO:1.

These and other embodiments of the subject invention will readily occur to those of skill in the art in view of the disclosure herein.

### BRIEF DESCRIPTION OF THE FIGURES

FIGS. 1A-1D show simultaneous, single tube sequencing of DNA and RNA. FIG. 1A shows a schematic of the SIMUL-SEQ method. FIG. 1B shows the average cross-species mapping rates for SIMUL-SEQ libraries produced from a mixture yeast of mRNA and human genomic DNA (n=2) as well as yeast RNA-seq (n=3) and human DNA-seq controls (n=2). Error bars depict standard deviations. FIG. 1C shows droplet digital PCR (ddPCR) assays on SIMUL-SEQ libraries (n=3 technical replicates/library) with varying amounts of RNA-specific PCR amplification followed by an additional 5 cycles of PCR with primer sets for both RNA and DNA. FIG. 1D shows DNA and RNA library ratios measured by ddPCR (n=3 technical replicates/library) are correlated with subsequent read ratios.

FIGS. 2A-2D show characterization of SIMUL-SEQ whole-genome data. FIG. 2A show coverage distributions for SIMUL-SEQ and DNA-seq genomes of the same individual. FIG. 2B shows Lorenz curves for the cumulative fraction of the covered genome versus the cumulative fraction of total mapped bases. Black line indicates theoretical limit for perfectly independent sampling. FIG. 2C shows a comparison of single nucleotide variant (SNV) calls between SIMUL-SEQ and DNA-seq genomes. (FIG. 2D) Comparison of insertion and deletions (indels) calls and size distributions between SIMUL-SEQ and DNA-seq genomes.

FIGS. 3A-3F show characterization of SIMUL-SEQ transcriptome data. FIGS. 3A and 3B show genomic distribution and strand specificity of SIMUL-SEQ RNA-indexed reads compared to RNA-seq control. SIMUL-SEQ DNA-indexed reads are included as a control. FIG. 3C shows the distribution of normalized transcript coverage for SIMUL-SEQ and RNA-seq transcriptome data. FIG. 3D shows the correlation between External RNA Controls Consortium (ERCC) spike-in control Log 10 RNA concentrations versus the average Log 10(RPKM) for SIMUL-SEQ (Spearman's ρ=0.97) and RNA-seq (Spearman's ρ=0.98) replicates (n=2). Note, zero RPKM values have been shifted to 1, and all ERCC transcripts are shown. FIG. 3E shows a pie chart of Ensembl genes (FPKM≧5) with noncoding biotypes from the SIMUL-SEQ transcriptome. FIG. 3F shows a scatter plot of Log 10(FPKM+1) values across all genes measured in the SIMUL-SEQ or RNA-seq datasets (Spearman's ρ=0.97).

FIGS. 4A-4C show comprehensive genome and transcriptome profiling of esophageal adenocarcinoma (EAC). FIG. 4A shows immunofluorescence staining (top) of fresh frozen EAC tissue with a simple epithelial marker (Keratin-8; orange) and Hoechst nuclear dye (blue). The tumor-stroma boundary is denoted with a white dashed line. Cresyl violet stained (bottom) serial section, with approximate tumor-stroma boundary denoted with a black dashed line. Scale bar is 100 μm. FIG. 4B shows coverage distributions for SIMUL-SEQ tumor genome and DNA-seq normal genomes. FIG. 4C shows a scatter plot for the average major allele frequencies for each transcript exhibiting allele-specific expression. Inset depicts reference and alterative RNA read counts for a known EGFR polymorphism.

FIGS. 5A and 5B show identification and biochemical characterization of a recurrent mutation in KIF3B. FIG. 5A shows a schematic of KIF3B locus, including positions of mutations found in targeted resequencing of 49 esophageal adenocarcinoma patients and 25 paired controls. Coverage for representative sample is shown. Note, the original sample that was subjected to the SIMUL-SEQ protocol was included as a positive control. FIG. 5B shows a histogram of the average (n=2) ATPase activity for recombinant wild-type and R293W mutant KIF3B motor domains when incubated with increasing quantities of microtubules. Activity was quantitated using an end-point measurement of free phosphate. Error bars depict standard deviations.

FIGS. 6A and 6B show SIMUL-SEQ library quality control. FIG. 6A shows a histogram of incubation times for parallel Tru-seq DNA and RNA library preparation as well as SIMUL-SEQ. FIG. 6B shows a high-sensitivity DNA bioanalyzer trace for a yeast/human mixed SIMUL-SEQ library. Note, this trace is representative of an average SIMUL-SEQ library.

FIGS. 7A and 7B show a comparison of variant calls between SIMUL-SEQ genome and DNA-seq replicates. FIGS. 7A and 7B show Venn diagrams comparing SNV (FIG. 7A) and indel (FIG. 7B) calls between the SIMUL-SEQ genome and two DNA-seq control genomes derived from different tissues of the same individual.

FIG. 8 shows the distribution of coding and noncoding genes in SIMUL-SEQ transcriptome. Bar graph of all Ensembl biotype annotations for genes with FPKM values greater than or equal to 5.

FIGS. 9A-9C show SIMUL-SEQ and RNA-seq replicates are well correlated. Scatter plots of Log 10(FPKM+1) gene measurements for SIMUL-SEQ (FIG. 9A) and RNA-seq (FIGS. 9B and 9C) replicates. Spear-man's ρ correlation values for each comparison are shown.

FIGS. 10A-10D show DNA and RNA sequencing data for 50,000 (50K) fibroblast replicates. FIG. 10A shows coverage distributions for SIMUL-SEQ libraries of the same individual. FIG. 10B shows a Venn diagrams comparing SNV calls between the SIMUL-SEQ replicates. FIG. 10C shows scatter plots of Log 10(FPKM+1) gene measurements for 50K SIMUL-SEQ replicates (Spearman's ρ=0.97). FIG. 10D shows a correlation between External RNA Controls Consortium (ERCC) spike-in control Log 10 RNA concentrations versus the average Log 10(RPKM+1) for SIMUL-SEQ (blue; Spearman's ρ=0.97) and 50K SIMUL-SEQ (orange; Spearman's ρ=0.96) fibroblast replicates (n=2/group). Note, zero values have been shifted to 1, and all ERCC transcripts are shown.

FIGS. 11A-11C show SIMUL-SEQ replicate and tumor RNA quality control analysis. FIGS. 11A and 11B show strand specificity and distribution of normalized transcript coverage for RNA-seq and SIMUL-SEQ replicates performed on fibroblasts as well as SIMUL-SEQ data obtained for esophageal adenocarcinoma tissue isolated using laser capture microscopy (SIMUL-SEQ EAC). FIG. 11C shows the fraction of reads mapping to various genomic annotations. Note, an increased intronic read fraction combined with a similar intergenic read fraction in the SIMUL-SEQ EAC sample likely indicates increased intron retention and/or a higher proportion of unspliced RNA in this specimen.

FIGS. 12A and 12B show purification of recombinant wild-type and R293W mutant motor domains. FIG. 12A shows a schematic of KIF3B protein, with motor domain and ATP binding region highlighted in dark gray and light gray, respectively. For biochemical assays, a region (1-365 amino acids) spanning KIF3B's motor domain was cloned and recombinantly expressed with an N-terminal 6×-Histidine tag (bottom). FIG. 12B shows a Coomassie stained gel of recombinant proteins pre- and post-induction with Isopropyl β-D-1-thiogalactopyranoside (IPTG) as well as after Ni²⁻ affinity purification.

### DETAILED DESCRIPTION OF THE INVENTION

The practice of the present invention will employ, unless otherwise indicated, conventional methods of molecular biology, cell biology, chemistry, and biochemistry within the skill of the art. Such techniques are explained fully in the literature. See, e.g., Next-generation Sequencing: Current Technologies and Applications (J. Xu ed., Caister Academic Press, 2014); High-Throughput Next Generation Sequencing: Methods and Applications (Methods in Molecular Biology, Y. M. Kwon and S. C. Ricke eds., Humana Press, 2011); Next Generation Sequencing: Translation to Clinical Diagnostics (L. C. Wong ed., Springer, 2013); T. Nolan and S. A. Bustin PCR Technology: Current Innovations (CRC Press, 3^{rd} edition, 2013); S. A. Bustin A-Z of Quantitative PCR (International University Line, 2004); E. van Pelt-Verkuil, A. van Belkum, J. P. Hays Principles and Technical Aspects of PCR Amplification (Springer, 2008); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

All publications, patents and patent applications cited herein, whether supra or infra, are hereby incorporated by reference in their entireties.

1. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a nucleic acid” includes a mixture of two or more such nucleic acids, and the like.

The term “about,” particularly in reference to a given quantity, is meant to encompass deviations of plus or minus five percent.

“Substantially purified” generally refers to isolation of a substance (compound, nucleic acid, polynucleotide, oligonucleotide, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides oligonucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a nucleic acid is a nucleic acid molecule devoid, in whole or part, of sequences normally associated with it in nature; or a sequence, as it exists in nature, but having heterologous sequences in association therewith; or a molecule disassociated from the chromosome.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

As used herein, the term “biological sample” includes any cell, tissue, or bodily fluid containing nucleic acids from a eukaryotic or prokaryotic organism, such as cells from plants, animals, fungi, protists, bacteria, or archaea. The biological sample may include cells from a tissue or bodily fluid, including but not limited to, blood, saliva, cells from buccal swabbing, fecal matter, urine, bone marrow, spinal fluid, lymph fluid, skin, organs, and biopsies, as well as in vitro cell culture constituents, including recombinant cells and tissues grown in culture medium. A biological sample may also include a viral particle comprising nucleic acids.

The term “common genetic variant” or “common variant” refers to a genetic variant having a minor allele frequency (MAF) of greater than 5%.

The term “rare genetic variant” or “rare variant” refers to a genetic variant having a minor allele frequency (MAF) of less than or equal to 5%.

The term “rare blood cell” refers to a type of cell found in blood in an amount of less than 100 cells/ml of whole blood.

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. A primer need not reflect the exact sequence of the template but must be sufficiently complementary to hybridize with a template. A primer may further comprise a “tail” comprising additional nucleotides at the 5′ end of the primer that are non-complementary to the template. Typically, the lengths of primers range between 7-100 nucleotides in length, such as 15-60, 20-40, and so on, more typically in the range of between 20-40 nucleotides in length, and any length between the stated ranges. Shorter primer molecules generally require cooler temperatures to form sufficiently stable hybrid complexes with the template. The term “primer site,” “priming site,” or “primer binding site” refers to the segment of the target DNA to which a primer hybridizes. Typically, a set of primers is used for amplification of a target polynucleotide, including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified.

The term “amplicon” refers to the amplified nucleic acid product of a PCR reaction or other nucleic acid amplification process (e.g., ligase chain reaction (LGR), nucleic acid sequence based amplification (NASBA), transcription-mediated amplification (TMA), Q-beta amplification, strand displacement amplification, or target mediated amplification).

As used herein, the term “probe” or “oligonucleotide probe” refers to a polynucleotide, as defined above, that contains a nucleic acid sequence complementary to a nucleic acid sequence present in the target nucleic acid analyte. The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally. Additionally, the oligonucleotide probe will typically be derived from a sequence that lies between the sense and the antisense primers when used in a nucleic acid amplification assay.

As used herein, the term “adapter” or “oligonucleotide adapter” refers to an oligonucleotide, as defined above, that is added to a nucleic acid (e.g., DNA or RNA) to facilitate high-throughput amplification and/or sequencing. Adapters may comprise, for example, a common priming site to allow massively parallel sequencing and/or amplification of DNA or cDNA in parallel with a set of universal primers. In addition, adapters may comprise indexing/barcoding sequences to identify the sample source (e.g., cell or tissue) from which each DNA or cDNA nucleic acid originated to allow pooling of DNA and cDNA derived from different samples for high-throughput amplification and/or sequencing. Alternatively or additionally, indexing/barcoding sequences can be used to distinguish nucleic acids derived from genomic DNA from nucleic acids (e.g., cDNA) derived from RNA to allow pooling of DNA and RNA from the same sample for high-throughput amplification and/or sequencing. For example, a “DNA index sequence” can be used to identify DNA sequences and an “RNA index sequence” can be used to identify RNA sequences. Adapters can be added to a nucleic acid, for example, by tagmentation, ligation, PCR, or any combination thereof.

A “barcode” or “index” sequence can include one or more nucleotide sequences that can be used to identify one or more particular nucleic acids (e.g., DNA or RNA). A barcode can comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more consecutive nucleotides.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

The term “universal,” when used in reference to nucleic acids, means a region of sequence that is common to two or more nucleic acid molecules where the molecules also have regions of different sequence. A universal sequence present in different members of a collection of molecules can allow the replication, amplification, sequencing, or detection of multiple different sequences using a single universal primer species that is complementary to the universal sequence. Thus, a universal primer includes a sequence that can hybridize specifically to a universal sequence.

A polynucleotide sequence “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from a polynucleotide comprising the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The “melting temperature” or “Tm” of double-stranded DNA is defined as the temperature at which half of the helical structure of DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25 degrees C. below the Tₘ. The Tₘ may be estimated using the following relationship: Tₘ=69.3+0.41(GC)% (Marmur et al. (1962) J. Mol. Biol. 5:109-118).

The terms “subject” refers to any invertebrate or vertebrate subject, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like, insects, nematodes, fish, amphibians, and reptiles. The term does not denote a particular age. Thus, both adult and newborn individuals are intended to be covered.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which may be used in the practice of the invention include, but are not limited to, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647,and Alexa Fluor 784, a cyanine dye such as Cy 3, Cy3.5, Cy5, Cy5.5, and Cy7, fluorescein, 2′,4′,5′,7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, NADPH, horseradish peroxidase (HRP), and α-β-galactosidase.

2. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Paired DNA and RNA profiling is increasingly employed in genomics research to uncover molecular mechanisms of disease and to explore personal genotype and phenotype correlations. The present invention is based on the discovery of an efficient method for simultaneously sequencing DNA and RNA (referred to as SIMUL-SEQ), which is capable of producing genome-wide DNA and RNA sequencing data of high quality from small quantities of cells or tissue (see Example 1). In particular, the inventors applied their SIMUL-SEQ method to microdissected esophageal adenocarcinoma tumor tissue in order to identify clinically relevant mutations associated with cancer progression. The use of SIMUL-SEQ revealed that the tumor had a highly aneuploid genome with extensive blocks of increased homozygosity with corresponding increases in allele-specific expression (see Example 1). The inventors further identified germline polymorphisms associated with responsiveness to cancer therapy and expressed mutations, including several known cancer genes as well as a recurrent mutation in the motor domain of KIF3B that significantly affects kinesin-microtubule interactions (see Example 1). Thus, the SIMUL-SEQ method can be used to generate comprehensive genomic and transcriptomic profiles from limited quantities of clinically relevant tissue and provide new biologic insights potentially useful for medical treatment.

In order to further an understanding of the invention, a more detailed discussion is provided below regarding the SIMUL-SEQ method and its various useful applications in scientific research, forensics, and medicine.

A. SIMUL-SEQ

In one aspect, the invention includes a method of simultaneously generating RNA and DNA sequencing libraries from the same sample. The method generally comprises: a) providing a biological sample comprising nucleic acids (e.g., DNA and RNA); b) isolating the nucleic acids from the sample; c) fragmenting the DNA by contacting the nucleic acids with a Class 2 transposase loaded with a transposable oligonucleotide adapter comprising a common priming site for DNA-specific amplification, wherein the transposase catalyzes cleavage of the DNA into a plurality of DNA fragments and ligation of the oligonucleotide adapter to each DNA fragment at the 5′ ends of its DNA strands; d) fragmenting the RNA by contacting the nucleic acids with RNase III, such that the RNase III cleaves the RNA into a plurality of RNA fragments; e) ligating a 3′ oligonucleotide adapter comprising a 3′ common priming site for RNA-specific amplification to the 3′ end of each RNA fragment using an RNA ligase; f) ligating a 5′ oligonucleotide adapter comprising a 5′ common priming site for RNA-specific amplification to the 5′ end of each RNA fragment using an RNA ligase; g) adding reverse transcriptase such that a plurality of cDNA fragments is produced from the plurality of RNA fragments; h) amplifying the plurality of DNA fragments with a set of DNA indexing primers that selectively bind to the common priming sites for DNA-specific amplification to produce a plurality of DNA amplicons, wherein each amplicon comprises a common priming site for DNA-specific sequencing and a DNA index sequence; i) amplifying the plurality of cDNA fragments with a set of RNA indexing primers that selectively bind to the common priming sites for RNA-specific amplification to produce a plurality of cDNA amplicons, wherein each amplicon comprises a common priming site for RNA-specific sequencing and an RNA index sequence; and j) sequencing the DNA amplicons and the cDNA amplicons, wherein the DNA index sequence is used to identify DNA sequences and the RNA index sequence is used to identify RNA sequences. All steps of the method may be performed with the RNA and DNA nucleic acids pooled together.

Nucleic acids may be obtained from any source. Sources of nucleic acid molecules include, but are not limited to, organelles, cells, tissues, organs, and organisms. For example, a biological sample containing nucleic acids to be analyzed can be any sample of cells, tissue, or fluid isolated from a prokaryotic or eukaryotic organism, including but not limited to, for example, blood, saliva, cells from buccal swabbing, fecal matter, urine, bone marrow, bile, spinal fluid, lymph fluid, sputum, ascites, bronchial lavage fluid, synovial fluid, samples of the skin, external secretions of the skin, respiratory, intestinal, and genitourinary tracts, tears, saliva, milk, organs, biopsies, and also samples of cells, including cells from bacteria, archaea, fungi, protists, plants, and animals as well as in vitro cell culture constituents, including recombinant cells and tissues grown in culture medium. The methods can be applied to living cells or fixed cells. In certain embodiments, the cell is an invertebrate cell, vertebrate cell, yeast cell, mammalian cell, rodent cell, primate cell, or human cell. Additionally, the biological sample may comprise a genetically aberrant cell, rare blood cell, or cancerous cell. A biological sample may also contain nucleic acids from viruses.

Cells may be pre-treated in any number of ways prior to amplification and sequencing of the DNA and RNA. For instance, in certain embodiments, the cell may be treated to disrupt (or lyse) the cell membrane, for example, by treating samples with one or more detergents (e.g., Triton-X-100, Tween 20, Igepal CA-630, NP-40, Brij 35, and sodium dodecyl sulfate) and/or denaturing agents (e.g., guanidinium agents). In cell types with cell walls, such as yeast and plants, initial removal of the cell wall may be necessary to facilitate cell lysis. Cell walls can be removed, for example, using enzymes, such as cellulases, chitinases, or bacteriolytic enzymes, such as lysozyme (destroys peptidoglycans), mannase, and glycanase. As will be clear to one of skill in the art, the selection of a particular enzyme for cell wall removal will depend on the cell type under study.

After lysing, nucleic acid extraction from cells may be performed using conventional techniques, such as phenol-chloroform extraction, precipitation with alcohol, or non-specific binding to a solid phase (e.g., silica). Care should be taken to avoid shearing the DNA and RNA during extraction steps. Additionally, enzymatic or chemical methods may be used to remove contaminating cellular components (e.g., ribosomal RNA, mitochondrial RNA, protein, or other macromolecules). For example, proteases can be used to remove contaminating proteins. A nuclease inhibitor may be used to prevent degradation of nucleic acids.

Removal of contaminating ribosomal RNA (rRNA), which constitutes about 95-98% of the RNA in a cell, improves detection of rare RNA species and the efficiency of transcriptome analysis. Depletion of rRNA can be accomplished in a variety of ways well-known in the art. For example, capture probes that bind to rRNA can be used to remove ribosomal RNA (e.g., RIBO-ZERO GOLD magnetic beads from Illumina Inc. (San Diego, Calif.) and RIBOMINUS capture probes from Thermo Fisher Scientific (Waltham, Mass.)). Alternatively, RNase H can be used to remove ribosomal RNA enzymatically (e.g., NEBNEXT rRNA depletion kit from New England Biolabs (Ipswich, Mass.) and RIBOGONE ribosomal RNA removal kit from Clontech Laboratories (Mountain View, Calif.)). In addition, when RNA sequences are amplified by reverse transcription polymerase chain reaction (RT-PCR) prior to sequencing, as discussed further below, judicious selection of primers that exclude amplification of ribosomal RNA can further minimize interference from rRNA.

Selective fragmentation of RNA can be accomplished by treatment of the nucleic acids with RNase III. After fragmentation, the RNA fragments are ligated to oligonucleotide adapters to facilitate high-throughput amplification. Oligonucleotide adapters comprising priming sites for RNA-specific amplification are ligated to the 3′ and 5′ ends of each RNA fragment using an RNA ligase. The addition of common 5′ and 3′ priming sites to each RNA fragment allows cDNA molecules, produced from the RNA fragments, to be amplified in parallel with a set of universal primers. The use of RNA-specific priming sites allows RNA to be amplified selectively in pooled nucleic acid mixtures containing DNA. Ligation can be accomplished with an RNA ligase such as T4 RNA ligase 1 or a genetically engineered variant thereof, which selectively ligates RNA-specific oligonucleotide adapters to the RNA fragments without modifying the DNA in pooled mixtures of the nucleic acids. The ability to ligate RNA-specific adapters selectively to the RNA fragments makes possible the production of a transcriptome sequencing library without physically separating the RNA from the DNA species.

Selective fragmentation and tagging of DNA (i.e., tagmentation) can be accomplished by treatment of the nucleic acids with a Class 2 transposase. Preferably, the transposase is hyperactive to allow efficient tagmentation. Hyperactive Tn5 transposases can be used in the practice of the invention and are commercially available from a variety of sources, including Illumina Inc. (San Diego, Calif.), Creative Biogene (Shirley, N.Y.), Epicentre Biotechnologies (Madison, Wis.), and Mandel Scientific (Ontario, Canada). Oligonucleotide adapters are complexed with the transposase to generate an active transposome. Transposome units insert randomly into a genomic template resulting in concerted fragmentation of the DNA and ligation of adapter oligonucleotide sequences to the generated fragments. The transposable oligonucleotide adapter comprises a common priming site for DNA-specific amplification to allow amplification of the generated DNA fragments using a set of universal DNA-specific primers. For a description of tagmentation and hyperactive transposases useful for carrying out the method, see, e.g., U.S. Pat. Nos. 9,080,211; 9,238,671; 6,294,385; 8,383,345; 9,040,256; 9,074,251; 7,083,980; and 8,829,171; U.S. Patent Application Publication No. 2015/0291942; and Brouilette et al. (2012) Dev. Dyn. 241(10):1584-1590; Petzke et al. (2009) Appl. Microbiol. Biotechnol. 83(5):979-986; Lyell et al. (2008) Appl. Environ. Microbiol. 74(22):7059-7063; Steiniger et al. (2006) Biochemistry 45(51):15552-15562; Steiniger-White et al. (2002) J. Mol. Biol. 322(5):971-982; Naumann et al. (2002) J. Bacteriol. 184(1):233-240; Twining et al. (2001) J. Biol. Chem. 276(25):23135-23143; Naumann et al. (2000) Proc. Natl. Acad. Sci. U.S.A. 97(16):8944-8949; York et al. (1998) Nucleic Acids Res. 26(8):1927-1933; and Goryshin et al. (1998) J. Biol. Chem. 273(13):7367-7374; herein incorporated by reference in their entireties.

The addition of adapters to the DNA and RNA fragments occurs in a mixed solution of the nucleic acids and does not require physical separation of the DNA and RNA nucleic acids in order to add the adapters. This method exploits the substrate specificity of the transposase and RNA ligase enzymes to selectively attach DNA-specific adapters to DNA and RNA-specific adapters to RNA, respectively in the pooled mixtures.

DNA and cDNA may be amplified prior to sequencing using any suitable polymerase chain reaction (PCR) technique known in the art. In PCR, a pair of primers is employed in excess to hybridize to the complementary strands of a target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

In particular, PCR uses relatively short oligonucleotide primers which flank the target nucleotide sequence to be amplified, oriented such that their 3′ ends face each other, each primer extending toward the other. Typically, the primer oligonucleotides are in the range of between 10-100 nucleotides in length, such as 15-60, 20-40 and so on, more typically in the range of between 20-40 nucleotides long, and any length between the stated ranges.

The polynucleotide sample is extracted and denatured, preferably by heat, and hybridized with first and second primers that are present in molar excess. Polymerization is catalyzed in the presence of the four deoxyribonucleotide triphosphates (dNTPs—dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. Preferably, PCR is carried out with a commercially available thermal cycler, e.g., Perkin Elmer.

RNA may be amplified by reverse transcribing RNA into cDNA with a reverse transcriptase, and then performing PCR (i.e., RT-PCR), as described above. Alternatively, a single enzyme may be used for both steps as described in U.S. Pat. No. 5,322,770, incorporated herein by reference in its entirety. In this manner, cDNA can be generated from all types of RNA, including mRNA, non-coding RNA, microRNA, siRNA, and viral RNA.

In certain embodiments, amplification comprises performing a clonal amplification method, such as, but not limited to bridge amplification, emulsion PCR (ePCR), or rolling circle amplification. In particular, clonal amplification methods such as, but not limited to bridge amplification, emulsion PCR (ePCR), or rolling circle amplification may be used to cluster amplified nucleic acids in a discrete area (see, e.g., U.S. Pat. No. 7,790,418; U.S. Pat. No. 5,641,658; U.S. Pat. No. 7,264,934; U.S. Pat. No. 7,323,305; U.S. Pat. No. 8,293,502; U.S. Pat. No. 6,287,824; and International Application WO 1998/044151 A1; Lizardi et al. (1998) Nature Genetics 19: 225-232; Leamon et al. (2003) Electrophoresis 24: 3769-3777; Dressman et al. (2003) Proc. Natl. Acad. Sci. USA 100: 8817-8822; Tawfik et al. (1998) Nature Biotechnol. 16: 652-656; Nakano et al. (2003) J. Biotechnol. 102: 117-124; herein incorporated by reference). For this purpose, additional adapter sequences (e.g., adapters with sequences complementary to universal amplification primers or bridge PCR amplification primers) suitable for high-throughput amplification may be added to DNA or cDNA fragments at the 5′ and 3′ ends. For example, bridge PCR primers, attached to a solid support, can be used to capture DNA templates comprising adapter sequences complementary to the bridge PCR primers. The DNA templates can then be amplified, wherein the amplified products of each DNA template cluster in a discrete area on the solid support.

In particular, the methods of the invention are applicable to digital PCR methods. For digital PCR, a sample containing nucleic acids is separated into a large number of partitions before performing PCR. Partitioning can be achieved in a variety of ways known in the art, for example, by use of micro well plates, capillaries, emulsions, arrays of miniaturized chambers or nucleic acid binding surfaces. Separation of the sample may involve distributing any suitable portion including up to the entire sample among the partitions. Each partition includes a fluid volume that is isolated from the fluid volumes of other partitions. The partitions may be isolated from one another by a fluid phase, such as a continuous phase of an emulsion, by a solid phase, such as at least one wall of a container, or a combination thereof In certain embodiments, the partitions may comprise droplets disposed in a continuous phase, such that the droplets and the continuous phase collectively form an emulsion.

The partitions may be formed by any suitable procedure, in any suitable manner, and with any suitable properties. For example, the partitions may be formed with a fluid dispenser, such as a pipette, with a droplet generator, by agitation of the sample (e.g., shaking, stirring, sonication, etc.), and the like. Accordingly, the partitions may be formed serially, in parallel, or in batch. The partitions may have any suitable volume or volumes. The partitions may be of substantially uniform volume or may have different volumes. Exemplary partitions having substantially the same volume are monodisperse droplets. Exemplary volumes for the partitions include an average volume of less than about 100, 10 or 1 μL, less than about 100, 10, or 1 nL, or less than about 100, 10, or 1 pL, among others.

After separation of the sample, PCR is carried out in the partitions. The partitions, when formed, may be competent for performance of one or more reactions in the partitions. Alternatively, one or more reagents may be added to the partitions after they are formed to render them competent for reaction. The reagents may be added by any suitable mechanism, such as a fluid dispenser, fusion of droplets, or the like.

After PCR amplification, nucleic acids are quantified by counting the partitions that contain PCR amplicons. Partitioning of the sample allows quantification of the number of different molecules by assuming that the population of molecules follows a Poisson distribution. For a description of digital PCR methods, see, e.g., Hindson et al. (2011) Anal. Chem. 83(22):8604-8610; Pohl and Shih (2004) Expert Rev. Mol. Diagn. 4(1):41-47; Pekin et al. (2011) Lab Chip 11 (13): 2156-2166; Pinheiro et al. (2012) Anal. Chem. 84 (2): 1003-1011; Day et al. (2013) Methods 59(1):101-107; herein incorporated by reference in their entireties.

Primers can be readily synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109. Poly(A) or poly(C), or other non-complementary nucleotide extensions may be incorporated into oligonucleotides using these same methods. Hexaethylene oxide extensions may be coupled to the oligonucleotides by methods known in the art. Cload et al., J. Am. Chem. Soc. (1991) 113:6324-6326; U.S. Pat. No. 4,914,210 to Levenson et al.; Durand et al., Nucleic Acids Res. (1990) 18:6353-6359; and Horn et al., Tet. Lett. (1986) 27:4705-4708.

Additionally, index or barcode sequences can be added to amplicon products to identify whether an amplicon was derived from DNA or RNA and the sample source from which each amplified nucleic acid originated. Index/barcode sequences can be used to distinguish amplicons derived from genomic DNA from amplicons derived from RNA (i.e., cDNA) to allow pooling of DNA and RNA from the same sample for high-throughput amplification and sequencing. For example, a “DNA index sequence” can be used to identify DNA sequences and an “RNA index sequence” can be used to identify RNA sequences. In one embodiment, the RNA index sequence comprises a nucleotide sequence of SEQ ID NO:1 or a nucleotide sequence having at least 95% identity to the nucleotide sequence of SEQ ID NO:1. Furthermore, the use of index/barcode sequences allows nucleic acids from different samples to be pooled in a single reaction mixture for sequencing while still being able to trace back a particular nucleic acid to the particular sample from which it originated. Each sample can be identified by a unique index/barcode sequence typically comprising at least five nucleotides. For example, each index/barcode sequence may be five, six, seven, or eight bases in length, or longer to differentiate between samples. An index/barcode sequence can be added during amplification by carrying out PCR with a primer that contains a region comprising the index/barcode sequence and a region that is complementary to an oligonucleotide adapter sequence (e.g., RNA-specific adapter attached to RNA fragment by ligation or DNA-specific adapter attached to DNA fragment by tagmentation) already ligated to the nucleic acid such that the index/barcode sequence is incorporated into the final amplified nucleic acid product. Index/barcode sequences can be added at one or both ends of an amplicon.

Moreover, oligonucleotides, particularly primer or probe oligonucleotides for amplification or sequencing, may be coupled to labels for detection. There are several means known for derivatizing oligonucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken et al., Nucl. Acids Res. (1978) 5:363-384 which discloses the use of ferritin-avidin-biotin labels; and Chollet et al., Nucl. Acids Res. (1985) 13:1529-1541 which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly, Nucl. Acids Res. (1987) 15:3131-3139, Gibson et al. Nucl. Acids Res. (1987) 15:6455-6467 and U.S. Pat. No. 4,605,735 to Miyoshi et al. Methods are also available for synthesizing sulfhydryl-derivatized oligonucleotides, which can be reacted with thiol-specific labels, see, e.g., U.S. Pat. No. 4,757,141 to Fung et al., Connolly et al., Nucl. Acids Res. (1985) 13:4485-4502 and Spoat et al. Nucl. Acids Res. (1987) 15:4837-4848. A comprehensive review of methodologies for labeling DNA fragments is provided in Matthews et al., Anal. Biochem. (1988) 169:1-25.

For example, oligonucleotides may be fluorescently labeled by linking a fluorescent molecule to the non-ligating terminus of the molecule. Guidance for selecting appropriate fluorescent labels can be found in Smith et al., Meth. Enzymol. (1987) 155:260-301; Karger et al., Nucl. Acids Res. (1991) 19:4955-4962; Guo et al. (2012) Anal. Bioanal. Chem. 402(10):3115-3125; and Molecular Probes Handbook, A Guide to Fluorescent Probes and Labeling Technologies, 11^{th} edition, Johnson and Spence eds., 2010 (Molecular Probes/Life Technologies). Fluorescent labels include fluorescein and derivatives thereof, such as disclosed in U.S. Pat. No. 4,318,846 and Lee et al., Cytometry (1989) 10:151-164. Dyes for use in the present invention include 3-phenyl-7-isocyanatocoumarin, acridines, such as 9-isothiocyanatoacridine and acridine orange, pyrenes, benzoxadiazoles, and stilbenes, such as disclosed in U.S. Pat. No. 4,174,384. Additional dyes include SYBR green, SYBR gold, Yakima Yellow, Texas Red, 3-(ε-carboxypentyl)-3′-ethyl-5,5′-dimethyloxa-carbocyanine (CYA); 6-carboxy fluorescein (FAM); CAL Fluor Orange 560, CAL Fluor Red 610, Quasar Blue 670; 5,6-carboxyrhodamine-110 (R110); 6-carboxyrhodamine-6G (R6G); N′,N′,N′,N′-tetramethyl-6-carboxyrhodamine (TAMRA); 6-carboxy-X-rhodamine (ROX); 2′,4′,5′,7′,-tetrachloro-4-7-dichlorofluorescein (TET); 2′,7′-dimethoxy-4′,5′-6 carboxyrhodamine (JOE); 6-carboxy-2′,4,4′,5′,7,7′-hexachlorofluorescein (HEX); Dragonfly orange; ATTO-Tec; Bodipy; ALEXA; VIC, Cy3, and Cy5. These dyes are commercially available from various suppliers such as Life Technologies (Carlsbad, Calif.), Biosearch Technologies (Novato, Calif.), and Integrated DNA Technolgies (Coralville, Iowa). Fluorescent labels include fluorescein and derivatives thereof, such as disclosed in U.S. Pat. No. 4,318,846 and Lee et al., Cytometry (1989) 10:151-164, and 6-FAM, JOE, TAMRA, ROX, HEX-1, HEX-2, ZOE, TET-1 or NAN-2, and the like.

Oligonucleotides can also be labeled with a minor groove binding (MGB) molecule, such as disclosed in U.S. Pat. No. 6,884,584, U.S. Pat. No. 5,801,155; Afonina et al. (2002) Biotechniques 32:940-944, 946-949; Lopez-Andreo et al. (2005) Anal. Biochem. 339:73-82; and Belousov et al. (2004) Hum Genomics 1:209-217. Oligonucleotides having a covalently attached MGB are more sequence specific for their complementary targets than unmodified oligonucleotides. In addition, an MGB group increases hybrid stability with complementary DNA target strands compared to unmodified oligonucleotides, allowing hybridization with shorter oligonucleotides.

Additionally, oligonucleotides can be labeled with an acridinium ester (AE) using the techniques described below. Current technologies allow the AE label to be placed at any location within the probe. See, e.g., Nelson et al., (1995) “Detection of Acridinium Esters by Chemiluminescence” in Nonisotopic Probing, Blotting and Sequencing, Kricka L. J. (ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

DNA or cDNA molecules may be further purified by immobilization on a solid support, such as silica, adsorbent beads (e.g., oligo(dT) coated beads or beads composed of polystyrene-latex, glass fibers, cellulose or silica), magnetic beads, or by reverse phase, gel filtration, ion-exchange, or affinity chromatography. Alternatively, an electric field-based method can be used to separate DNA/cDNA fragments from other molecules. Exemplary electric field-based methods include polyacrylamide gel electrophoresis, agarose gel electrophoresis, capillary electrophoresis, and pulsed field electrophoresis. See, e.g., U.S. Pat. Nos. 5,234,809; 6,849,431; 6,838,243; 6,815,541; and 6,720,166; and Sambrook et al., Molecular Cloning: A Laboratory Manual (3^{rd} Edition, 2001); Recombinant DNA Methodology (Selected Methods in Enzymology, R. Wu, L. Grossman, K. Moldave eds., Academic Press, 1989); and J. Kieleczawa DNA Sequencing II: Optimizing Preparation And Cleanup (Jones & Bartlett Learning; 2^{nd} edition, 2006); herein incorporated by reference in their entireties.

B. Sequencing of Nucleic Acids

The selective attachment of DNA-specific and RNA-specific adapters to the nucleic acids allows the DNA and cDNA (derived from RNA) molecules to be pooled and sequenced simultaneously. Any high-throughput technique for sequencing can be used in the practice of the invention. DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like. These sequencing approaches can thus be used to sequence the DNA and RNA (cDNA) fragments.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). Such methods may comprise amplifying the isolated molecules either before or after they are spatially isolated on a solid surface. Prior amplification may comprise emulsion-based amplification, such as emulsion PCR, or rolling circle amplification.

Of particular interest is sequencing on the Illumina HiSeq or MiSeq platforms, which use reversible-terminator sequencing by synthesis technology (see, e.g., Shen et al. (2012) BMC Bioinformatics 13:160; Junemann et al. (2013) Nat. Biotechnol. 31(4):294-296; Glenn (2011) Mol. Ecol. Resour. 11(5):759-769; Thudi et al. (2012) Brief Funct. Genomics 11(1):3-11; herein incorporated by reference).

Once an RNA or DNA sequencing library, created according to the methods described herein, has been sequenced, the sequencing data can be processed to assemble the raw short nucleic acid sequences (or short reads) into longer nucleic acid sequences (long reads). In certain embodiments, overlapping sequence reads are assembled into contigs or full or partial contiguous sequences of a nucleic acid of interest (e.g. DNA gene, intergenic regulatory region, or RNA coding or non-coding transcript) by sequence alignment, computationally or manually, whether by pairwise alignment or multiple sequence alignment of overlapping sequence reads. Recognition of RNA-specific adapter sequences is used to identify and assemble RNA sequences, whereas recognition of DNA-specific adapter sequences is used to identify and assemble DNA sequences. Thus, the use of the adapter sequences avoids cross-contamination between RNA and DNA sequences during assembly.

Sequencing reads can be trimmed to remove the known adapter sequences. A number of programs are available for this purpose including, but not limited to, Cutadapt (pypi.python.org/pypi/cutadapt), Trimmomatic (usadellab.org/cms/?page=trimmomatic), Skewer (biomedcentral.com/1471-2105/15/182), the FASTX-toolkit (hannonlab.cshl.edu/fastx_toolkit/), Scythe (github.com/vsbuffalo/scythe), and others.

C. Applications

The methods of the invention will find numerous applications in basic research and development. The technology described herein makes possible combined genome-wide genomic and transcriptomic profiling and detailed study of relationships between genotype and phenotype. A major advantage of the technology is the ability to produce both DNA and RNA sequence data from the same cell populations with the nucleic acids pooled together at every step of library preparation, which eliminates complications from sample variation and differences in sample handling that can confound data analysis. A further advantage of this method is that less material is required for preparation of “matched” DNA and RNA libraries because only a single sample is needed. The methods disclosed herein will find use in various applications in scientific research, medicine, and forensics that benefit from genotyping and expression profiling.

The methods of the invention can be used to detect common or rare genetic events, such as mutations (e.g., nucleotide substitutions, insertions, or deletions) and alterations of copy number. Mutations can be common genetic variants or rare genetic variants and may include single nucleotide variants, gene fusions, translocations, inversions, duplications, frameshifts, missense, nonsense, or other mutations associated with a phenotype of interest (e.g., disease or condition). The methods of the invention can also be used to obtain information about the distribution of genomic and transcriptomic variation in a particular population.

The methods of the invention will be especially useful for detecting genomic and transcriptomic changes associated with physiological processes and diseases. For example, certain diseased cells or tissues may have genetic differences from normal or healthy cells and corresponding changes in gene regulation and allele expression. Therefore, combined genomic and transcriptomic analysis of such diseased cells or tissues may be useful for diagnosing a disease and detecting changes that may be associated with disease progression. Furthermore, detecting such changes associated with a disease may be useful for identifying potential therapeutic targets for treatment.

In particular, the methods of the invention can be used to analyze genomic and transcriptomic variation in tumors, including mutations and copy number variation associated with regulatory variation and expression of aberrant proteins leading to cancer progression.

D. Kits

The above-described reagents, including a Class 2 transposase (e.g., hyperactive Tn5 transposase), oligonucleotide adapters (e.g., adapter comprising a common priming site for DNA-specific amplification, a 5′ adapter comprising a 5′ common priming site for RNA-specific amplification, a 3′ oligonucleotide adapter comprising a 3′ common priming site for RNA-specific amplification), RNase III, RNA ligase, reverse transcriptase, DNA polymerase (e.g., Taq polymerase for PCR), DNA indexing PCR primers; and RNA indexing PCR primers can be provided in kits with suitable instructions and other necessary reagents in order to carry out preparation of RNA and DNA sequencing libraries as described above. The kit will normally contain in separate containers the various primers, adapters, and enzymes, and other reagents required to carry out the method. Instructions (e.g., written, CD-ROM, DVD, flash drive, SD card, digital download etc.) for preparing RNA and DNA sequencing libraries simultaneously as described herein usually will be included with the kit. The kit may also contain other packaged reagents and materials (e.g., wash buffers, nucleotides, silica spin columns, capture probes for ribosomal RNA depletion, and other reagents and/or devices for performing e.g., clonal amplification, digital PCR, NGS sequencing, ribosomal RNA depletion, nucleic acid purification, and the like).

3. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Comprehensive Genome and Transcriptome Profiling Using Simultaneous DNA and RNA Sequencing (SIMUL-SEQ)

Introduction

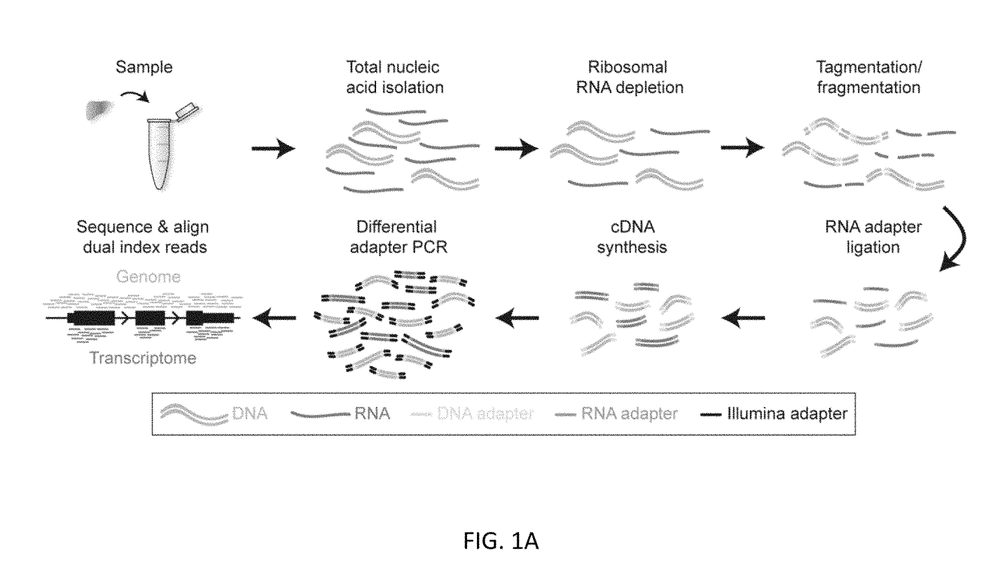

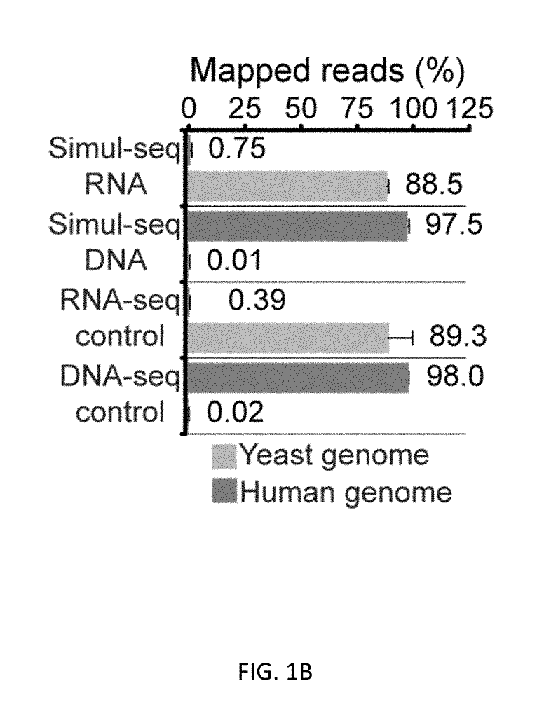

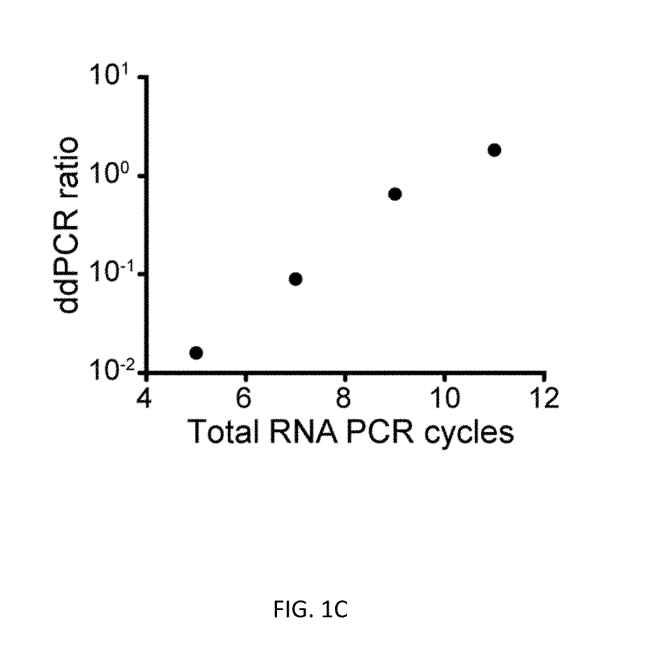

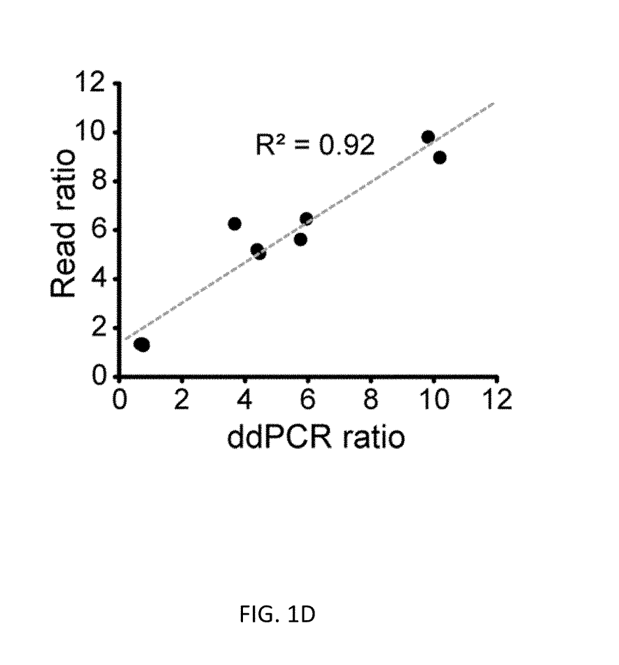

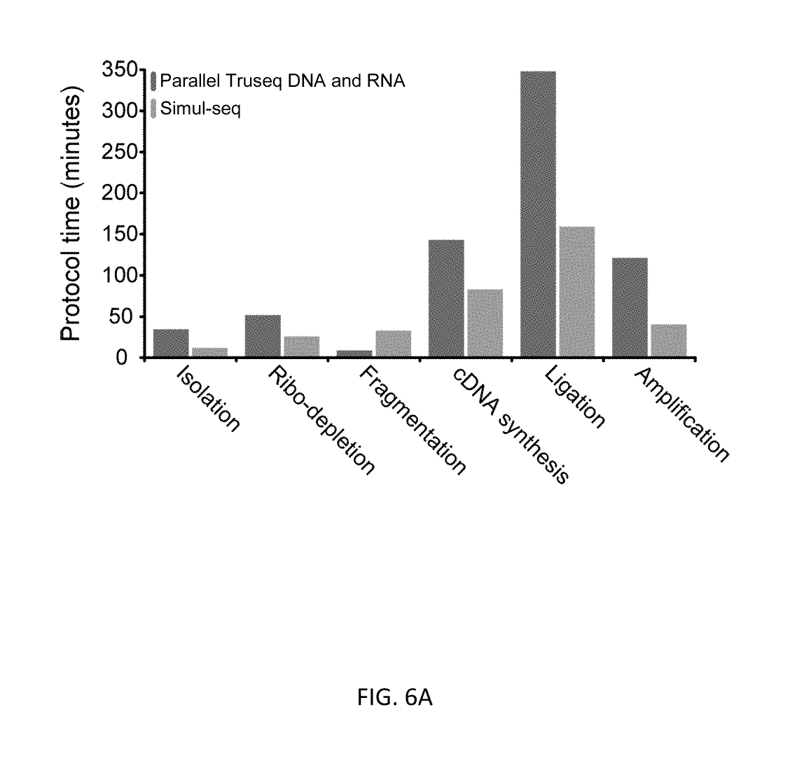

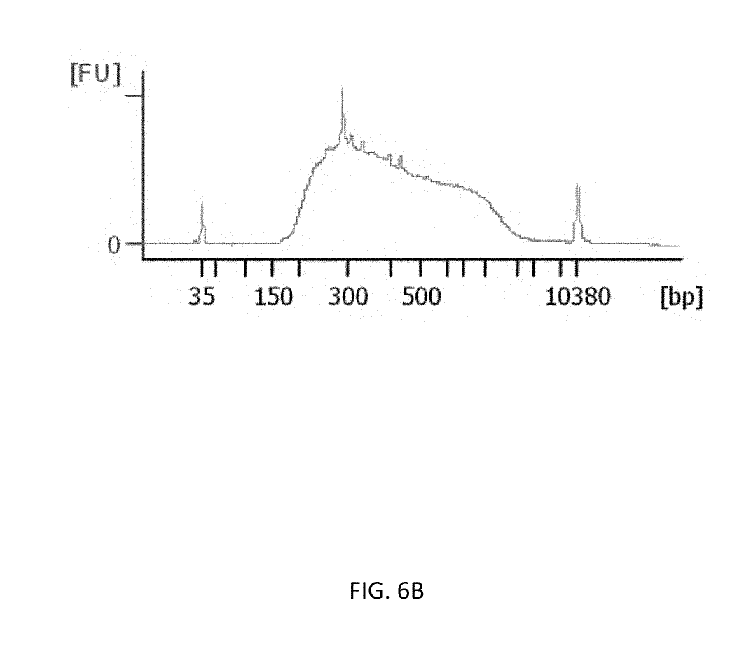

Here, we report a dual DNA and RNA sequencing method that streamlines the workflow while retaining the lower bias and technical variability of standard independent sequencing approaches. Our simultaneous DNA and RNA sequencing method (SIMUL-SEQ) leverages the enzymatic specificities of the Tn5 transposase and RNA ligase to produce whole-genome and transcriptome libraries without physical separation of the nucleic acid species (FIG. 1A), reducing the library preparation time when compared to standard independent library approaches (FIG. 6A). SIMUL-SEQ also employs ribosomal depletion rather than poly-A enrichment, thereby maintaining many biologically relevant classes of noncoding RNAs and decreasing 3′ bias for samples with lower RNA quality (Adiconis et al. (2013) Nat. Methods 10:623-629, Zhao et al. (2014) BMC Genomics 15:419). Additionally, SIMUL-SEQ incorporates dual 5′ and 3′ indices specific for both DNA and RNA molecules, minimizing cross contamination due to spurious ligation and tagmentation or from template switching during pooled PCR. Finally, differential amplification from distinct RNA and DNA adapter sequences can be used to adjust the read outputs derived from either library.

Results

SIMUL-SEQ Efficiently Produces Distinct RNA-Seq and DNA-Seq Data

To rigorously assess the specificity of the SIMUL-SEQ method, we first produced libraries derived from a mixture of 50 ng of human genomic DNA and 100 ng of yeast mRNA (FIG. 6B). We quantified the presence of both DNA-seq and RNA-seq libraries in the pool using droplet digital PCR (ddPCR). Subsequent sequencing and alignment of the dual-indexed reads to the yeast and human genomes revealed cross-species mapping rates that were similar to those observed in yeast RNA-seq and human DNA-seq libraries produced independently (FIG. 1B), indicating that the SIMUL-SEQ method specifically barcodes the DNA and RNA with distinct adapters. Next, we leveraged these adapters to optimize read outputs for various applications and starting material inputs using differential PCR. To verify this approach, we tested increasing numbers of cycles of PCR with RNA-specific primers followed by a constant number of cycles with both DNA and RNA primer sets. Inclusion of RNA-specific cycles increased the fraction of the total library derived from RNA, as measured by ddPCR (FIG. 1C). Moreover, ddPCR quantification of the DNA and RNA constituents prior to sequencing was also highly correlated with subsequent read outputs (FIG. 1D), enabling users to perform quality control on the mixed libraries before high throughput sequencing.

SIMUL-SEQ DNA Sequencing Data is of High Quality

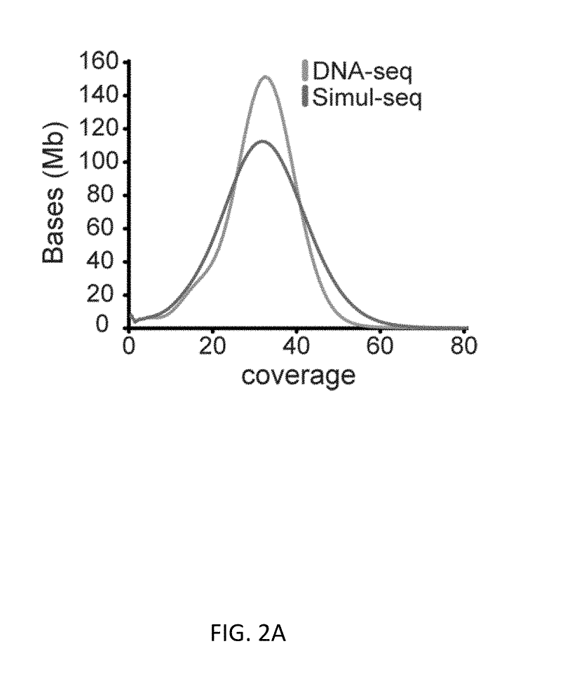

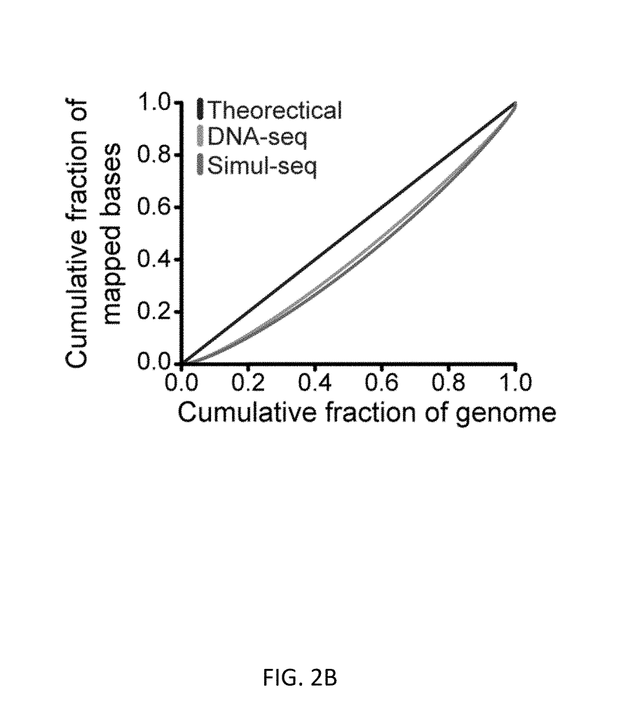

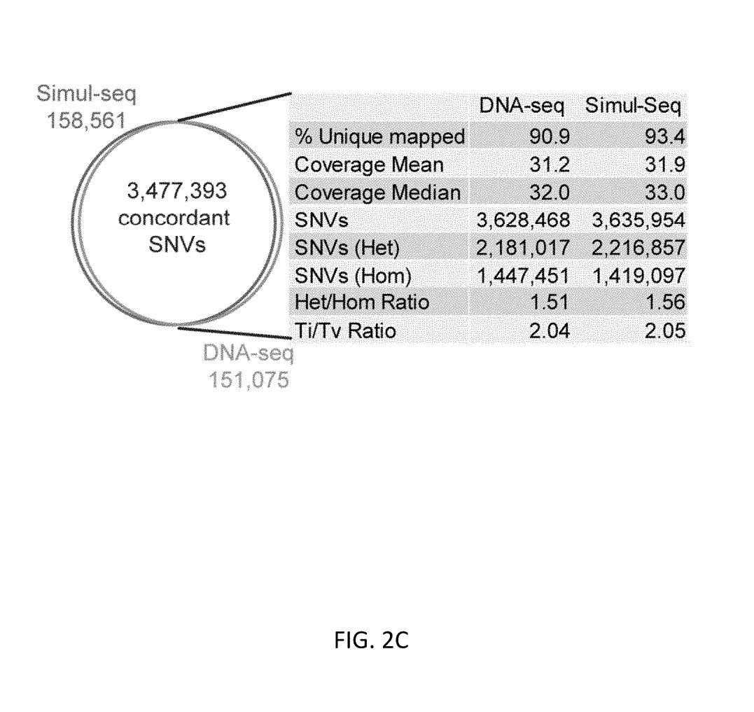

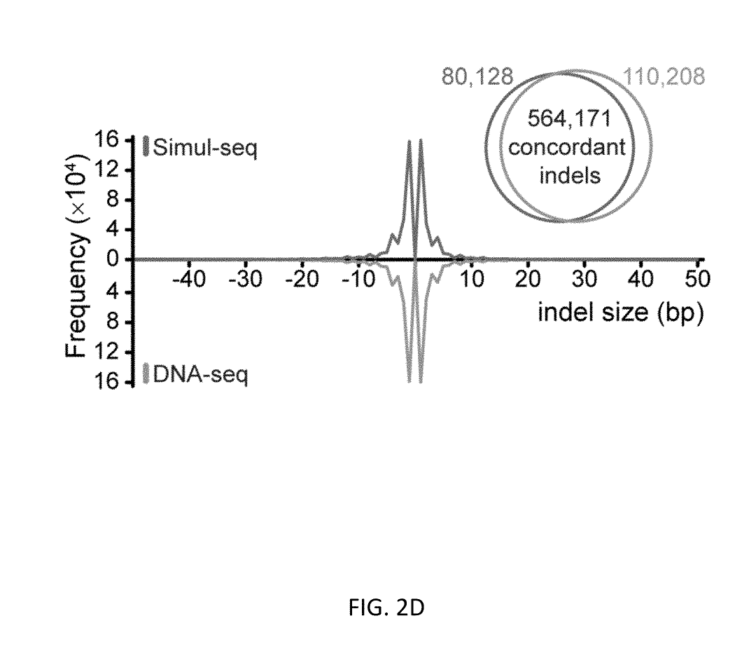

To benchmark SIMUL-SEQ against established library preparation methods, we next applied the approach to fibroblasts derived from an individual who had previously been subjected to whole-genome sequencing (Lam et al. (2011) Nat. Biotechnol. 30:78-82). In parallel, we also prepared independent RNA-seq libraries from these cells using an analogous RNA ligase-based protocol. For the SIMUL-SEQ library, we obtained 560,218,621 and 57,091,162 dual-indexed DNA and RNA 101 bp paired-end reads, respectively (Table 2). Ninety-three percent of SIMUL-SEQ DNA reads mapped to the genome, producing an average genomic depth of 31.9× (FIG. 2A). Although the SIMUL-SEQ coverage distribution was consistent with the distribution obtained from a library previously generated using an established DNA-seq method (Lam et al., supra) (FIG. 2A), the distribution exhibited some sequencing bias characteristic of the Tn5 transposase (Adey et al. (2010) Genome Biol. 11:R119). To further explore potential coverage biases, we generated Lorenz curves comparing the cumulative fraction of mapped bases with the cumulative fraction of the genome covered. Both the SIMUL-SEQ and the DNA-seq control genomes exhibited comparable read distributions (FIG. 2B), indicating that pooled DNA and RNA library preparation and sequencing does not introduce sequencing bias in excess of standard methods.

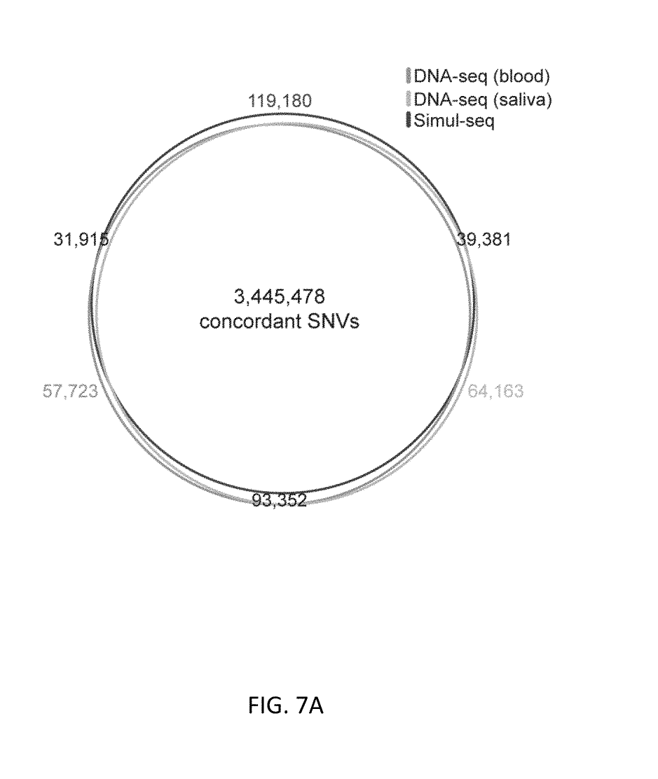

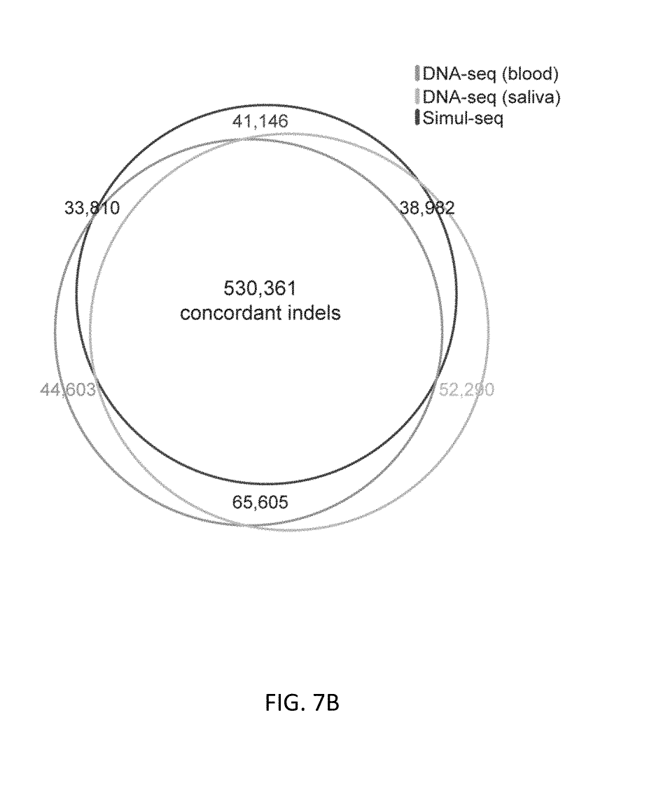

Whole-genome sequencing is generally performed to identify variants polymorphic among populations or associated with disease. Therefore, we next compared variant calls between the SIMUL-SEQ and control DNA-seq genomes. Of the 3,635,954 single nucleotide variants (SNVs) determined in the SIMUL-SEQ genome, 95.6% were concordant with SNVs called in the standard DNA-seq genome (FIG. 2C). In addition, the identity and size distribution of small insertions and deletions (indels) identified in the SIMUL-SEQ genome were similar to those obtained from the DNA-seq genome, with 87.5% of SIMUL-SEQ-derived indels exhibiting concordance with the standard genome (FIG. 2D). These degrees of concordance were comparable to those observed from previously published biologic replicates using a standard DNA-seq approach (Lam et al., supra) (FIGS. 7A, 7B), demonstrating that SIMUL-SEQ produces high quality whole-genome data.

SIMUL-SEQ RNA Sequencing Data is of High Quality

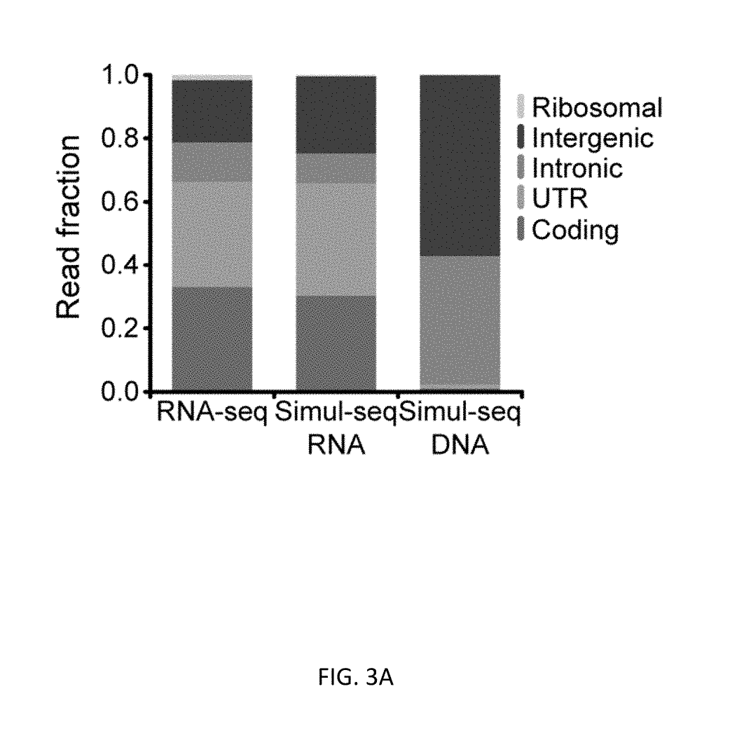

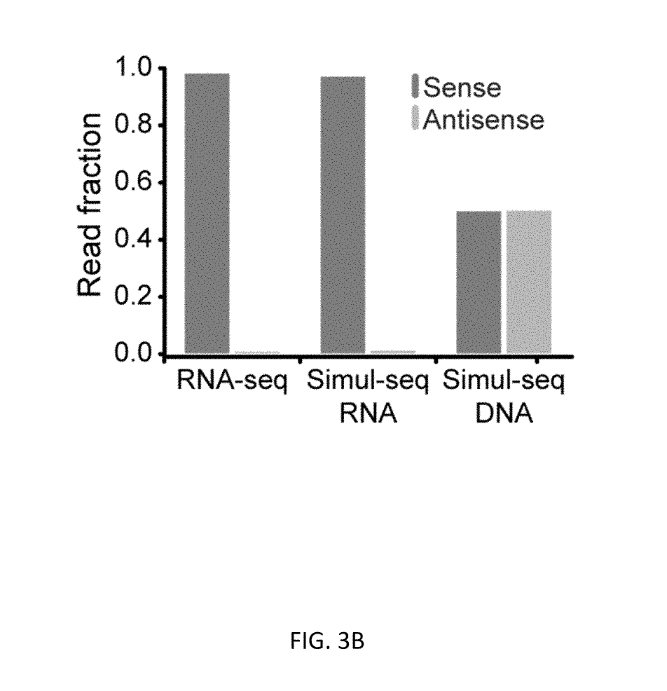

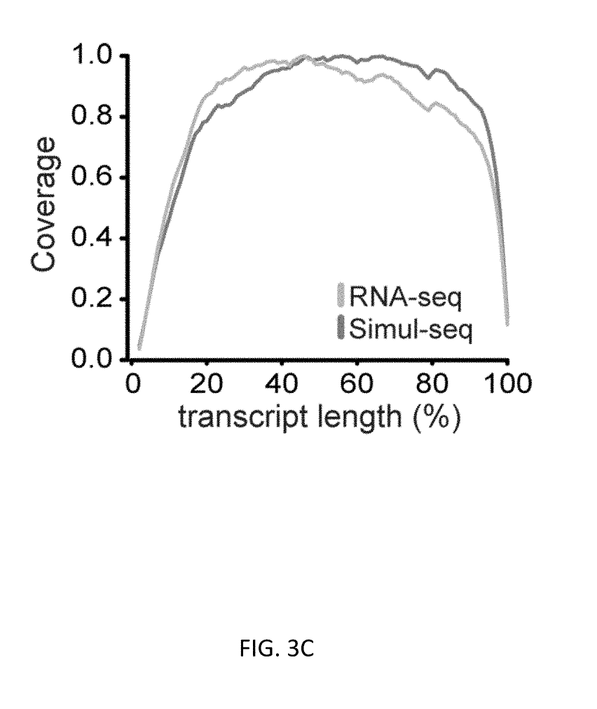

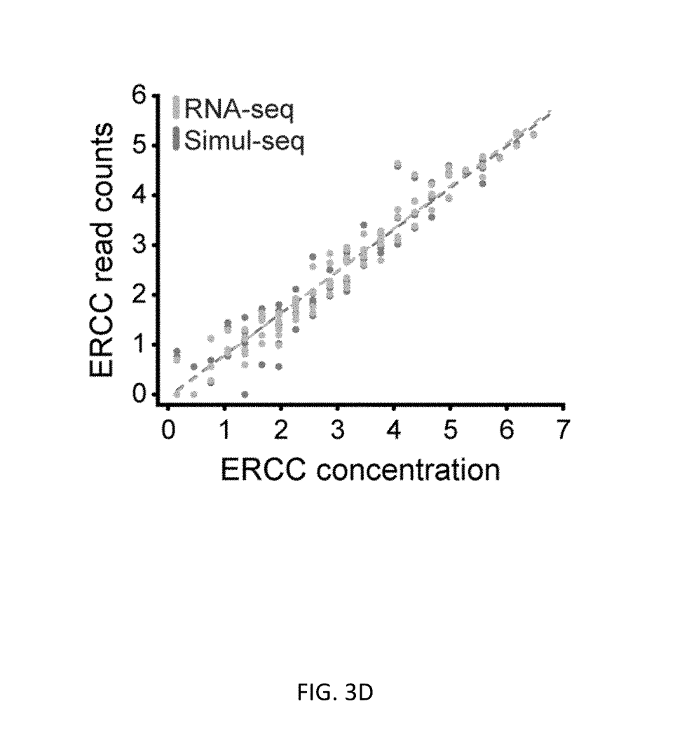

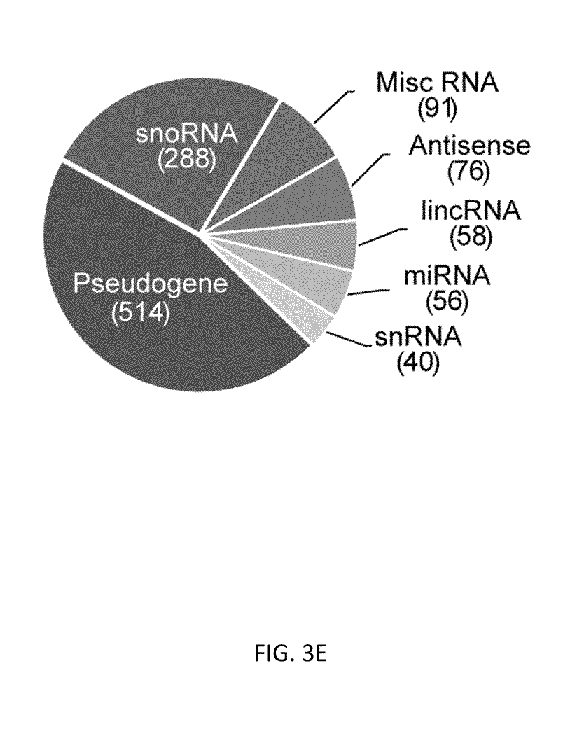

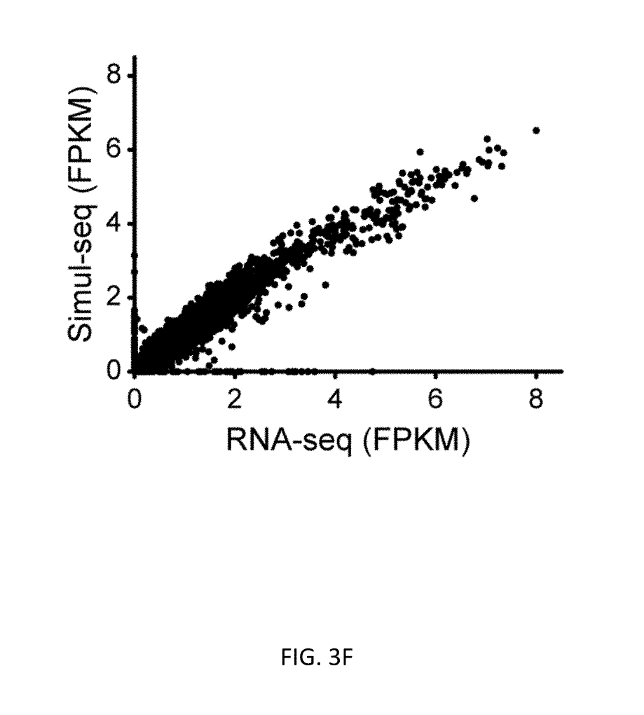

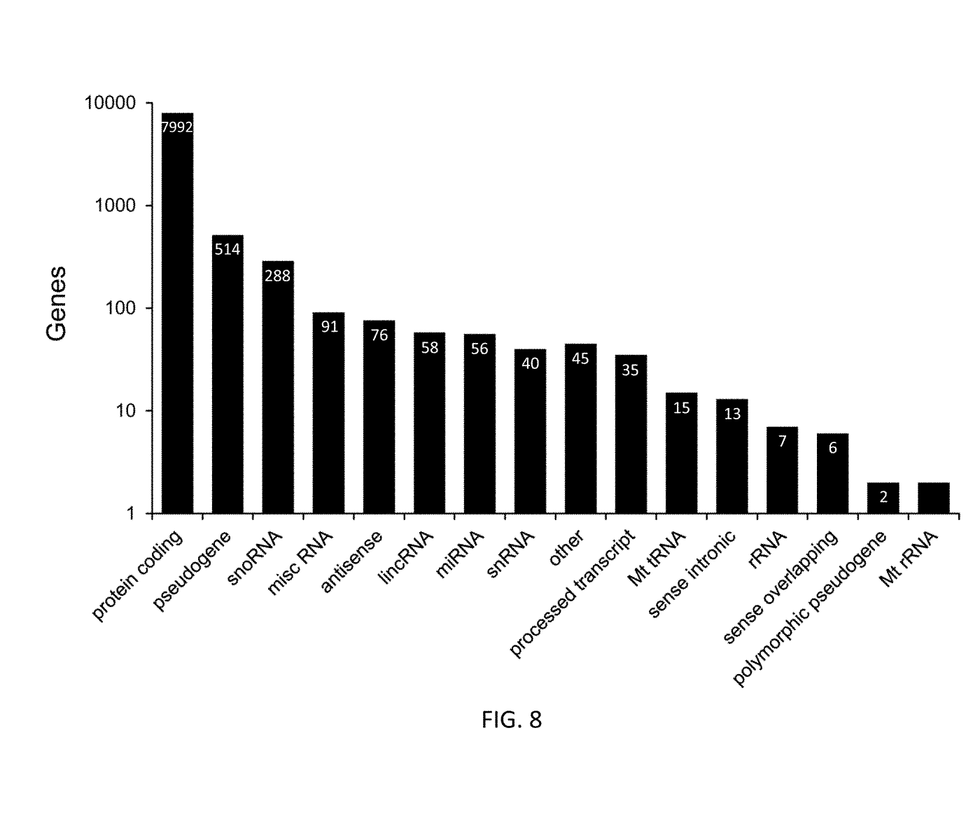

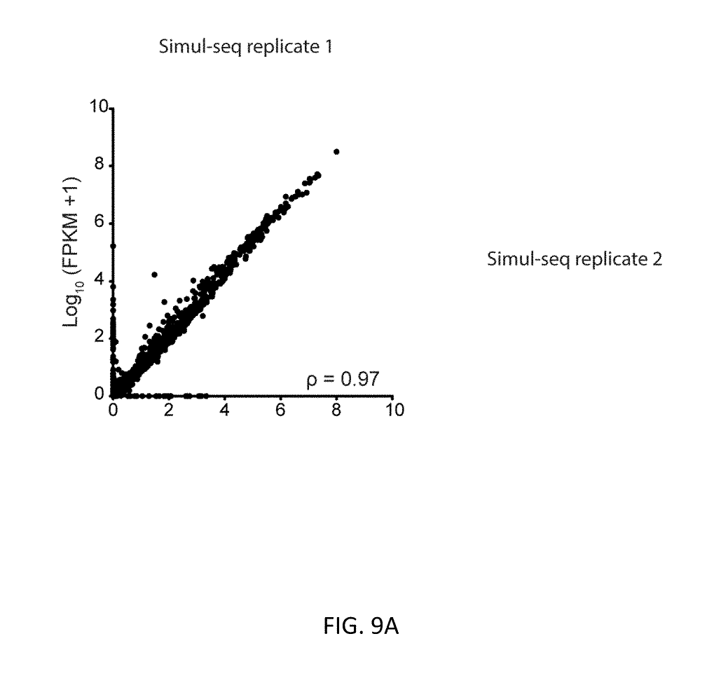

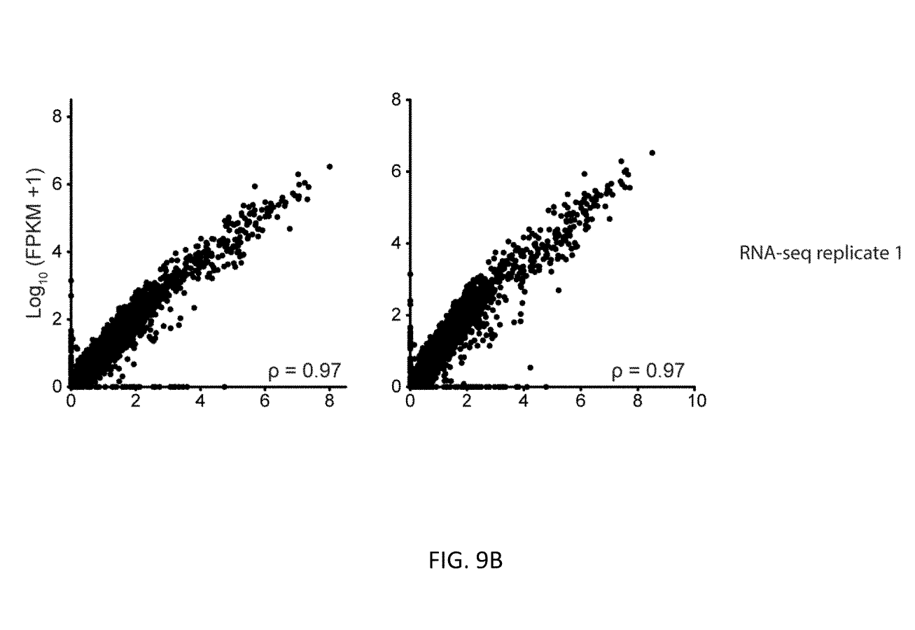

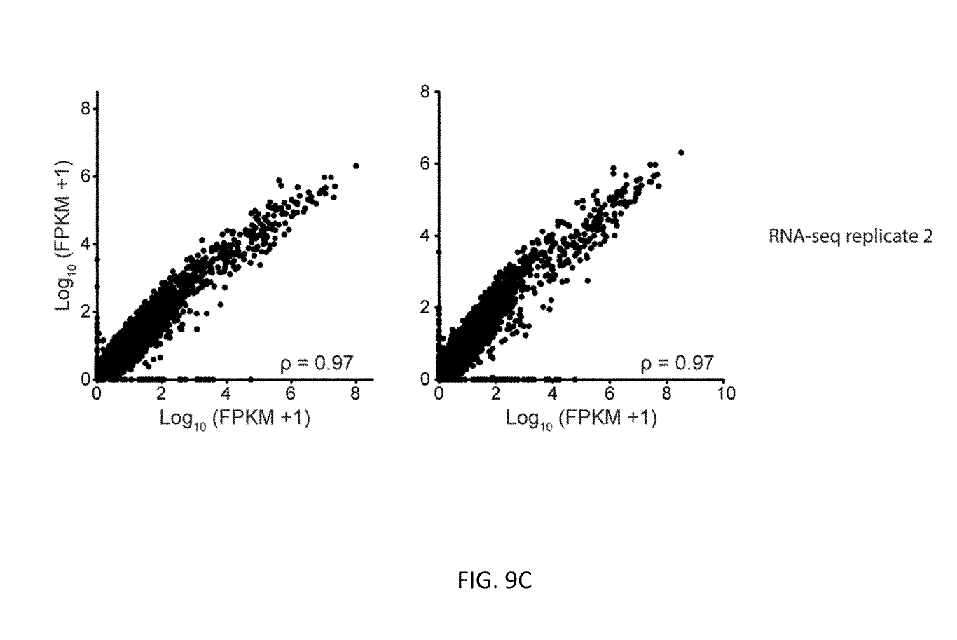

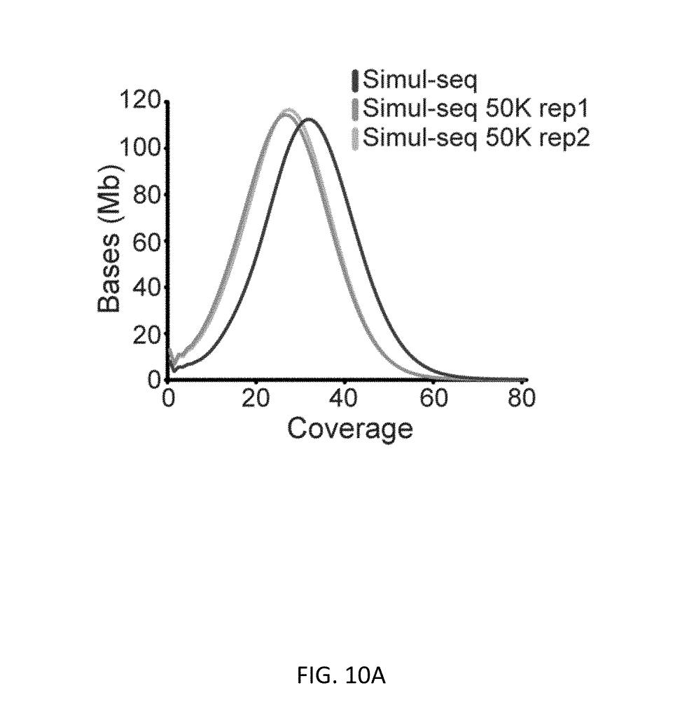

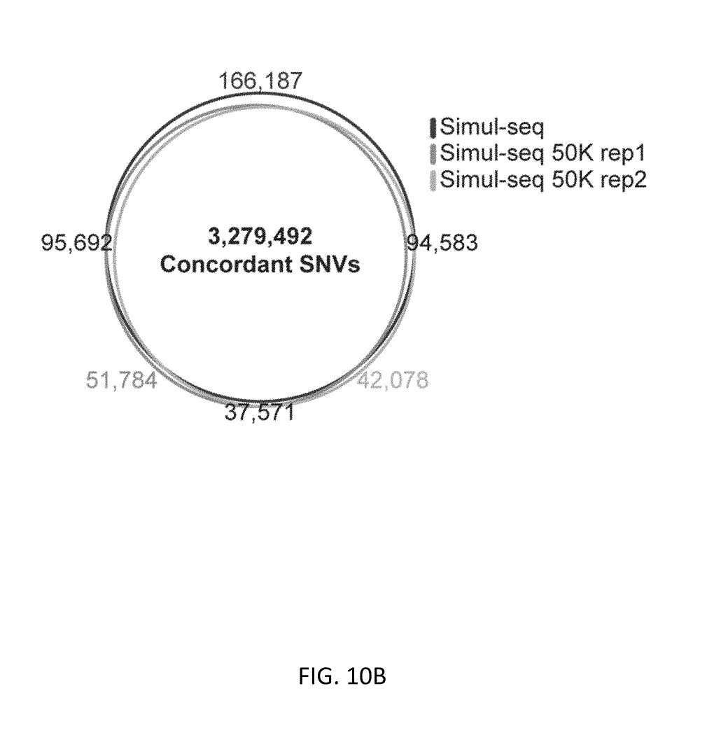

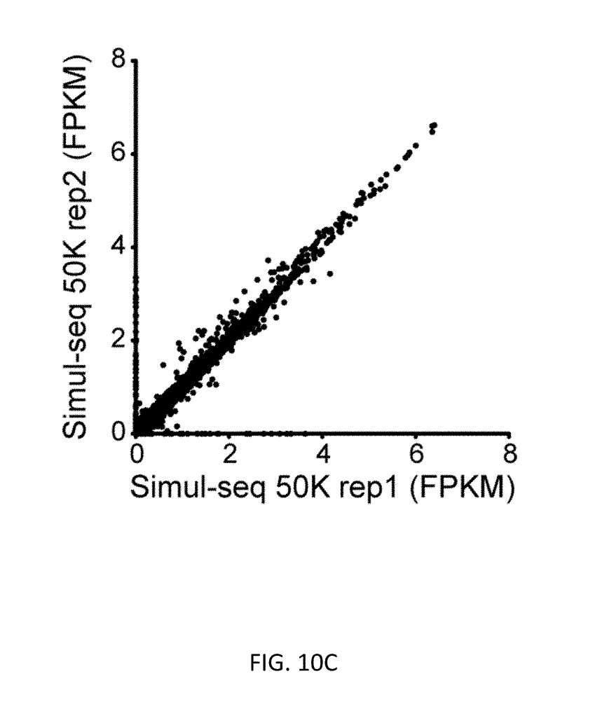

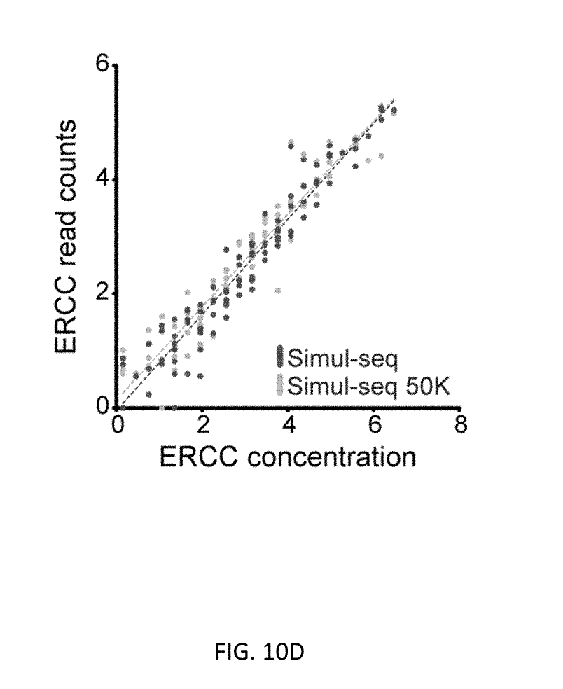

Next, we examined the quality of the RNA sequencing data. Similar to RNA-seq control data, SIMUL-SEQ RNA reads were effectively depleted for ribosomal sequences and mapped primarily to transcribed regions of the genome (FIG. 3A). SIMUL-SEQ RNA reads were also highly strand-specific and evenly distributed across the length of transcripts (FIGS. 3B, 3C), enabling accurate transcriptome quantification and isoform analysis. As a control, SIMUL-SEQ DNA reads mapped primarily to intronic and intergenic regions of the genome and were evenly distributed between each DNA strand, as expected (FIGS. 3A, 3B). To rigorously assess the technical variation of transcript quantification, External RNA Controls Consortium (ERCC) RNA standards (Baker et al. (2005) Nat. Methods 2:731-734) were spiked into the total nucleic acid mixture. SIMUL-SEQ produced ERCC transcript measurements that were both highly correlated with the known ERCC concentrations as well as RNA-seq control ERCC measurements (FIG. 3D). The SIMUL-SEQ-derived transcriptome contained 7,992 protein-coding genes as well as an additional 1,123 noncoding genes that would be largely not detected with poly-A enrichment (FIG. 3E, FIG. 8). Moreover, transcriptome-wide FPKM measurements were both reproducible and well correlated with RNA-seq control FPKMs (FIG. 3F, FIG. 9). Taken together, these experiments demonstrate that the SIMUL-SEQ protocol efficiently produces high-quality whole-genome sequencing data and RNA sequencing data, allowing for the comprehensive profiling of genomic and transcriptomic variation from the same cell population. In addition, we have applied the method to as few as 50,000 fibroblasts, obtaining coverage distributions and variant calls (FIGS. 10A, 10B) as well as FPKM and ERCC expression data (FIGS. 10C, 10D) that were both reproducible and well correlated with our previous results.

Application of SIMUL-SEQ to Cancer

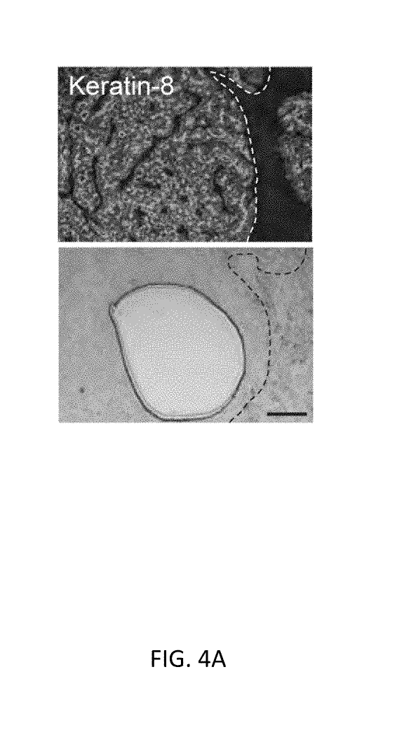

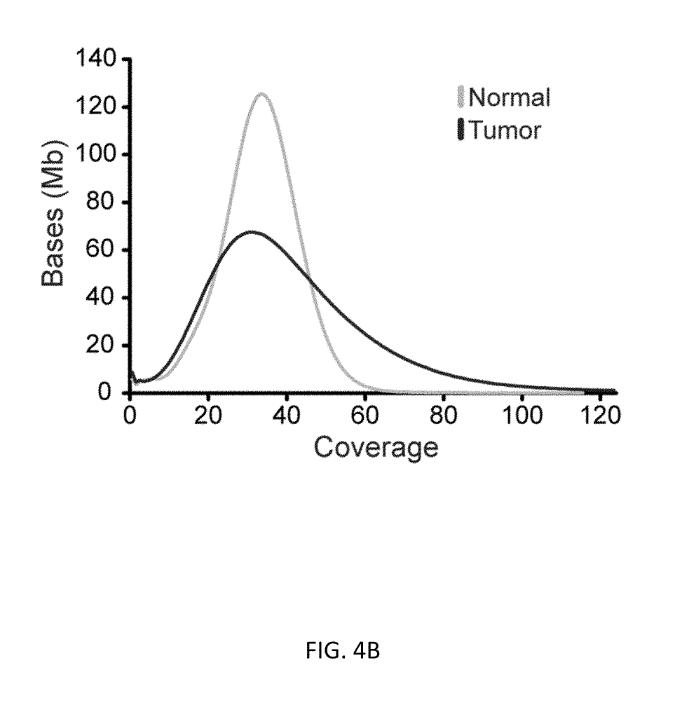

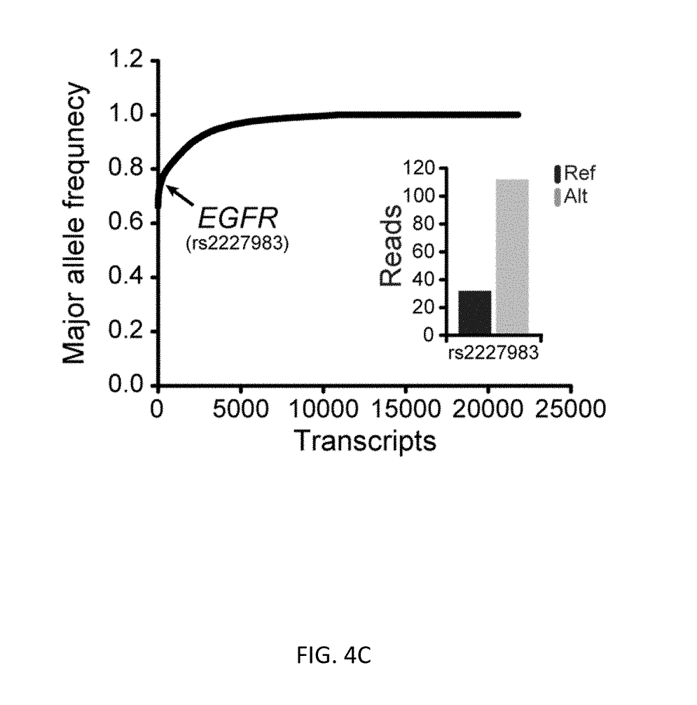

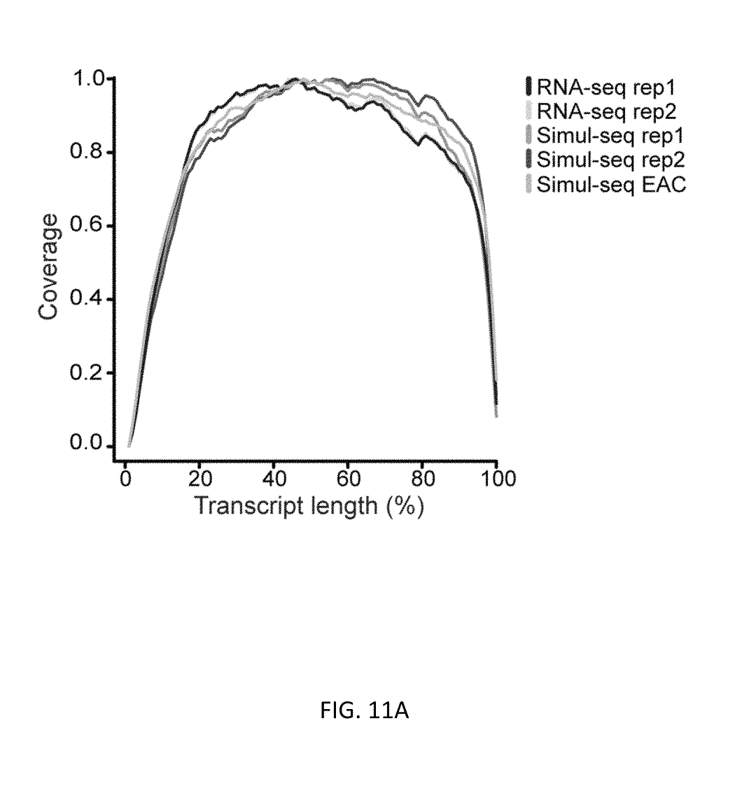

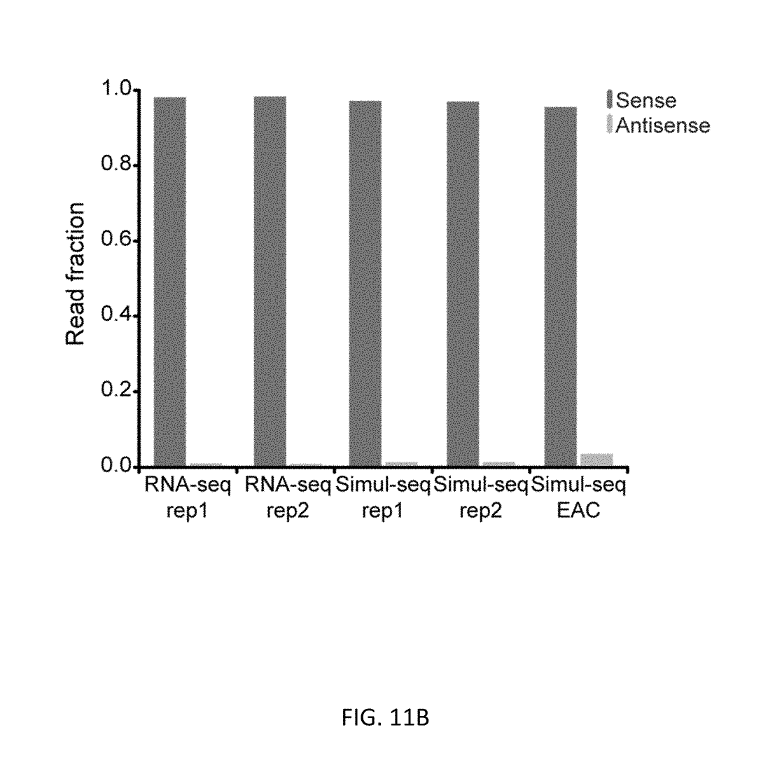

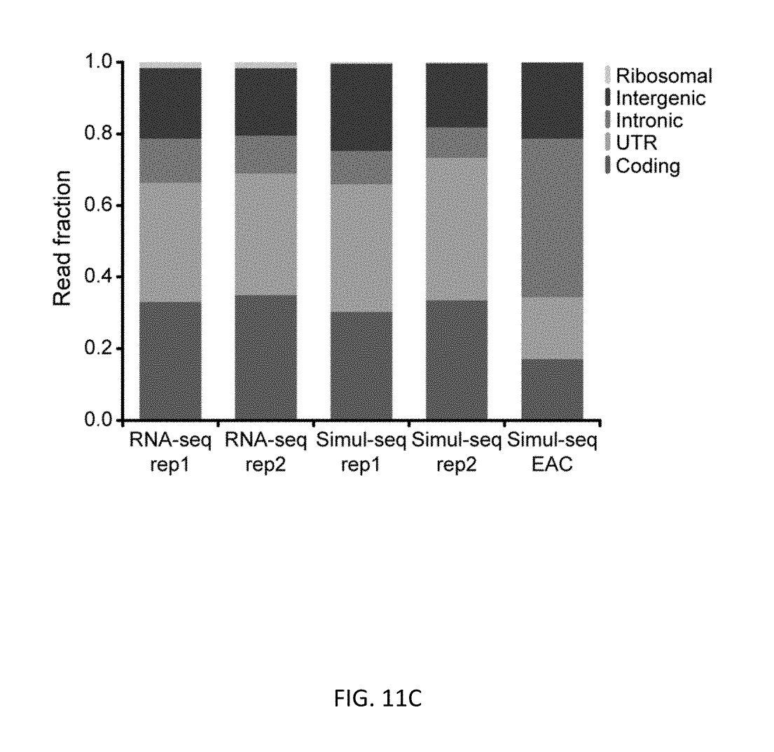

Integrative DNA and RNA profiling is increasingly employed in cancer genomics to distinguish driver mutations of various types (e.g., protein coding, regulatory, structural variants, etc.) from the multitude of passenger mutations (Shah et al., supra; Lawrence et al. (2014) Nature 505:495-501, Weinstein et al. (2014) Nature 507:315-322). To test SIMUL-SEQ in this tissue context, we applied the method to laser capture microdissected material (˜150 μg) isolated from a male subject with metastatic esophageal adenocarcinoma (EAC) (FIG. 4A). Deep sequencing of the SIMUL-SEQ EAC library produced 727,341,682 DNA and 191,398,961 RNA 101 bp dual-indexed paired-end reads, with 95.1% and 79.4% of the reads mapping to the genome and transcriptome, respectively (Table 2). Similar to the data acquired from fibroblasts, the SIMUL-SEQ RNA reads primarily mapped to transcribed regions, were highly strand-specific and evenly distributed over transcripts (FIGS. 11A-11C). However, the percentage reads mapping to introns was increased for this library, suggesting an increased rate of intron-retention and/or number of unspliced transcripts in this tumor specimen. The tumor genome was sequenced to an average coverage of 38× and displayed a skewed coverage distribution indicative of large-scale copy number alterations (FIG. 4B).

Comparing the SIMUL-SEQ tumor genome with a DNA-seq paired normal genome revealed a highly aneuploid genomic landscape, with somatic evidence for 142 structural variants and 9 expressed gene fusions as well as 15,607 SNVs and 2,904 indels. Globally, the ratio of heterozygous to homozygous SNPs for the tumor genome was 0.49, an exceptional deviation from the typically observed ratio of ˜1.5 (FIG. 2C). This low ratio appears to be driven by extensive blocks of loss of heterozygosity (LOH) and is consistent with an early whole-genome duplication event followed by acquired uniparental allelic loss. Analysis of allele-specific expression using the SIMUL-SEQ EAC transcriptome data provided further support for extensive LOH, with 92.9% of the identified allele-specific transcripts exhibiting average major allele frequencies of greater than or equal to 0.9 (FIG. 4C). Given the high levels of LOH-induced ASE in the tumor, we hypothesized that damaging germline variants in tumor suppressor genes might be specifically expressed in the tumor. Indeed, we identified 8 nonsynonymous variants in tumor suppressor genes (as defined by the TSGene 2.0 database (Zhao et al. (2016) Nucleic Acids Res. 44(D1):D1023-1031)) where a PolyPhen-2 (Adzhubei et al. (2010) Nat. Methods 7:248-249)—and SIFT (Kumar et al. (2009) Nat. Protoc. 4:1073-1081)—predicted damaging allele was predominantly expressed.

To distill the 15,607 somatic SNVs into potential oncogenic mutations, we first identified 80 nonsynonymous mutations, which we subsequently refined to 29 expressed mutations by leveraging the SIMUL-SEQ RNA reads (Table 1). In addition to identifying potential driver mutations, these expressed protein altering mutations also represent possible neoantigens from which patient-specific immunotherapies may be derived (Yadav et al. (2014) Nature 515:572-576, Robbins et al. (2013) Nat. Med. 19a:747-752, Schumacher et al. (2015) Science 348(6230):69-74). Notably, three Cosmic Cancer census genes (Coin et al. (2004) Nat. Rev. Cancer 4:177-183) (TP53, ATM and ESWR1) were found to harbor expressed somatic missense mutations. While ESWR1 is typically a constituent of an oncogenic fusion protein and the R45W mutation in the ATM serine/threonine kinase tumor suppressor is not yet characterized, the Y220C mutation is a known TP53 hotspot that decreases protein stability (Joerger et al. (2006) Proc. Natl. Acad. Sci. U.S.A. 103:15056-15061, Bullock et al. (2000) Oncogene 19:1245-1256). Moreover, we found that the TP53 locus exclusively expressed the damaging allele (Table 1), exacerbating the loss of TP53 function and likely underpinning the widespread genomic instability observed in this tumor specimen. Interestingly, this patient also exhibited ASE for common germline polymorphisms in the epidermal growth factor receptor gene (EGFR, rs2227983) as well as the cyclin D1 gene (CCND1, rs9344) that are associated with response to chemotherapeutic treatments (Gautschi et al. (2006) Lung Cancer 51:303-311, Absenger et al. (2014) Pharmacogenomics J. 14:130-134, Gonçalves et al. (2008) BMC Cancer 8:169, Hsieh et al. (2012) Cancer Sci. 103:791-796). For example, the EGFR variant allele was primarily expressed (FIG. 4C) and has been linked with enhanced response to anti-EGFR immunotherapy in colorectal cancer (Gonçalves et al., supra; Hsieh et al., supra).

Characterization of a Recurrent Mutation in a Kinesin Family Gene

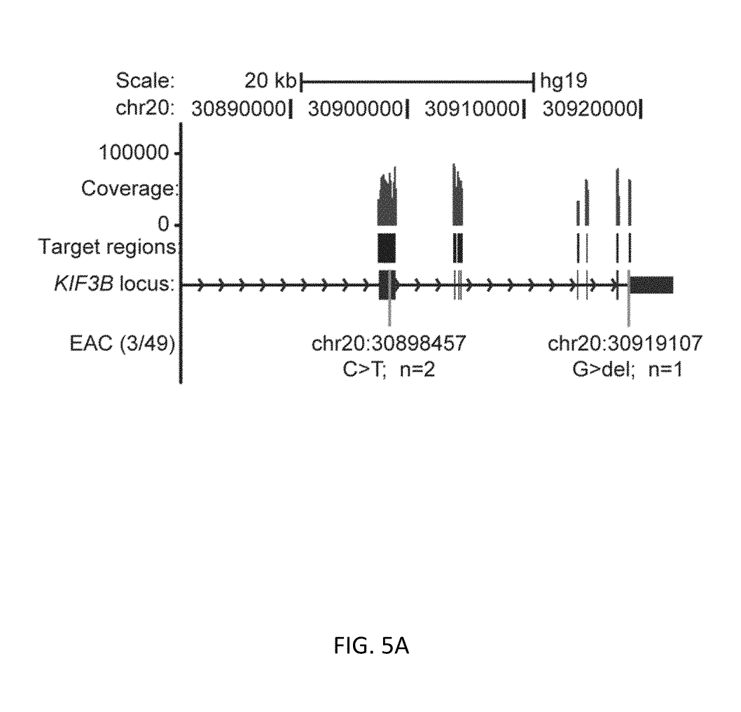

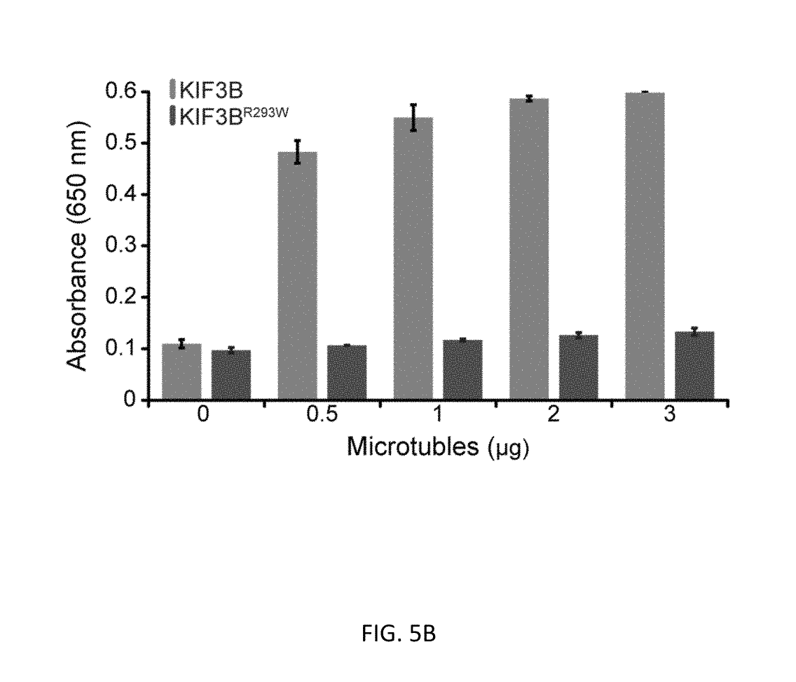

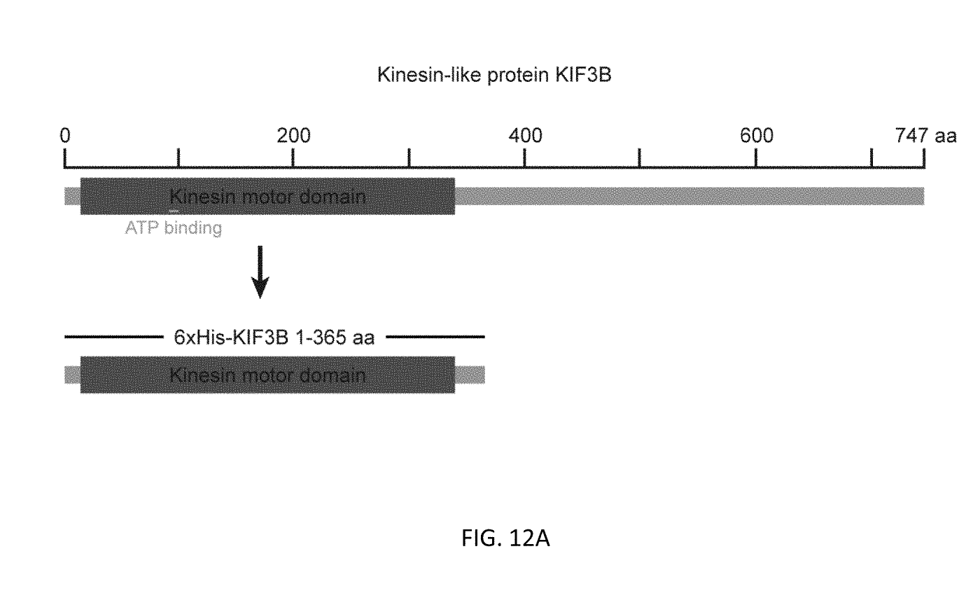

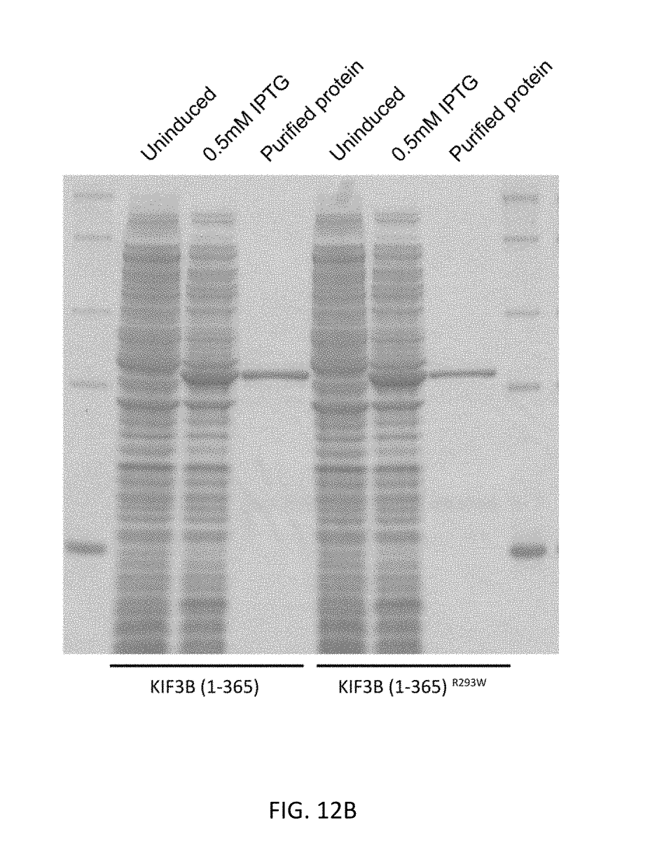

In addition to discovering clinically relevant alterations in known cancer genes, we observed an expressed arginine to tryptophan mutation in KIF3B (R293W), a type II kinesin motor protein. Although several kinesin family members have established roles in cancer (Yu et al. (2010) Cancer 116:5150-60), KIF3B somatic coding mutations have not been previously described. However, KIF3B has been linked to the intracellular trafficking of several tumor suppressor genes (Yu et al., supra; Jimbo et al. (2002) Nat. Cell Biol. 4(4):323-327), and biochemical data has shown that substitution of specific arginine and lysine residues within the kinesin motor domain negatively impacts kinesin-microtubule association (Woehlke et al. (1997) Cell 90:207-216). To further explore KIF3B mutation frequency in EAC, we performed targeted resequencing of the KIF3B locus in a cohort of 49 esophageal adenocarcinoma samples, with 25 matched normals. Overall, KIF3B harbored verified nonsynonymous mutations in ˜6% of the tumor samples, and the R293W mutation was observed in a second independent patient (FIG. 5A). To investigate the functional consequences of this recurrent R293W mutation, we purified recombinant wild-type and mutant KIF3B motor domains (FIGS. 12A, 12B). When compared to wild-type, the mutant motor domain displayed a significantly reduced rate of ATP hydrolysis upon incubation with various concentrations of microtubules, suggesting that the R293W mutation abrogates kinesin-microtubule binding (FIG. 5B). Together, these results demonstrate the benefits of SIMUL-SEQ in providing comprehensive DNA and RNA datasets, leading to the annotation of several clinically important variants as well as the description of a functionally significant recurrent mutation.

Discussion

As sequencing technologies advance and more individuals are profiled in both clinical and research settings, straightforward methods for generating comprehensive and accurate whole-genome and transcriptome sequencing data will become increasingly valuable. Currently, DNA and RNA sequencing datasets are largely produced in parallel from independent cell populations. However, the combined sequencing of both nucleic acid species from single cells was recently enabled by the development of two methods, DR-seq (Dey et al. (2015) Nat. Biotechnol. 33:285-289) and G&T-seq (Macaulay et al. (2015) Nat. Methods 12(6):519-522). While these new dual sequencing approaches allow high-resolution investigation into cellular heterogeneity, they also suffer from the coverage gaps and increased technical variability intrinsic to single-cell analyses. SIMUL-SEQ provides a complementary approach that focuses on producing comprehensive DNA and RNA profiles from limited quantities of tissues or cells rather than single cells. By leveraging enzyme specificities to barcode both nucleic acid species, our method also generates a single pooled library for sequencing, which both reduces the library preparation time and keeps paired genome and transcriptome datasets physically linked. Importantly, whereas DR- and G&T-seq depend upon polyadenylation to distinguish RNA transcripts from genomic DNA, the use of RNA-ligase in SIMUL-SEQ allows for a ribosomal RNA depletion step. Therefore, SIMUL-SEQ retains biologically and clinically important noncoding RNA species and may reduce sensitivity to starting RNA quality. We demonstrated that SIMUL-SEQ produces whole-genome data that has low bias and variant calls concordant with control genomes sequenced to an equivalent depth. Similarly, transcript measurements, coverage and strandedness were all well correlated between SIMUL-SEQ libraries and RNA-seq controls. These experiments show that SIMUL-SEQ libraries are comparable to standard DNA and RNA sequencing libraries produced independently, enabling comprehensive genotype and phenotype comparisons in a single workflow.

Cancer genome interpretation is one scenario where integration of precise and comprehensive DNA and RNA landscapes has proven useful but can be challenging due to limited starting material. Moreover, tumor heterogeneity increases the likelihood of discrepancies between genome and transcriptome profiles prepared in parallel on separate cell populations. Therefore, we applied SIMUL-SEQ to ˜150 μg of laser capture microdissected tumor tissue, revealing a highly aneuploid tumor genome with several interesting biologic characteristics. Among the 29 expressed nonsynonymous SNVs present in this EAC genome, we discovered a recurrent R293W mutation in the plus-end-directed motor protein KIF3B that dramatically reduced kinesin-microtubule interaction. Although a statistically significant increase in mutation rate for KIF3B has not been reported in large-scale cancer exome sequencing studies, including esophageal adenocarcinoma (Agrawal et al. (2012) Cancer Discov. 2(10):899-905, Dulak et al. (2013) Nat. Genet. 45:478-86), these efforts are still largely underpowered (Lawrence et al. (2014) Nature 505:495-501). Overall, we found that ˜6% of the examined EAC samples harbored protein altering mutations in KIF3B, a frequency consistent with recently published data from whole-genome sequencing of 22 EACs (Nones et al. (2014) Nat. Commun. 5:5224). Intriguingly, overexpression of c-terminal truncations of KIF3B induced aneuploidy in NIH3T3 cells (Haraguchi et al. (2006) J. Biol. Chem. 281:4094-4099). Moreover, KIF3B has been linked to the intracellular trafficking of several tumor suppressors, including the adenomatous polyposis coli (APC) as well as the von Hippel-Lindau (VHL) protein (Jimbo et al., supra; Yu et al., supra). Although further experiments are necessary to delineate specific functional roles for KIF3B mutation in esophageal tumorigenesis, our work demonstrates the utility of SIMUL-SEQ to provide the comprehensive profiling necessary for disease variant discovery.

In addition to this novel KIF3B mutation, we also identified a number of clinically relevant variants in this EAC patient sample. We observed a known TP53 hotspot mutation (Y220C) combined with additional loss of expression of the wild-type allele. This Y220C mutation has been shown to markedly destabilize the TP53 protein at body temperatures (Bullock et al. (2000) Oncogene 19:1245-56) but is also a target of several small molecules designed to restore TP53 function in tumors (Joerger et al., supra; Liu et al. (2013) Nucleic Acids Res. 41:6034-6044). TP53 inactivation followed by whole-genome duplication and chromosomal catastrophe is a frequent trajectory for EAC development (Nones et al., supra; Stachler et al. (2015) Nat. Genet. 47:1047-1055) and is consistent with our observations for this tumor. Among the wide-spread LOH induced by this genomic instability, we also detected ASE for germline variants with pharmacogenomic links to the efficacy of several cancer therapies used in EAC. The EGFR polymorphism (rs2227983) observed in this patient is associated with increased survival of colorectal cancer patients treated with Cetuximab (Gonçalves et al. (2008) BMC Cancer 8:169, Hsieh et al. (2012) Cancer Sci. 103:791-796), perhaps via attenuation of EGFR pathway signaling (Moriai et al. (1994) Proc. Natl. Acad. Sci. U.S.A. 91:10217-10221). In contrast, the patient harbored a second variant in CCND1 (rs9344) that is inversely correlated with overall survival in colorectal cancer patients treated with Cetuximab (Zhang et al. (2006) Genomics 16, 475-483). In both cases, however, the beneficial allele was predominantly expressed in the tumor, suggesting a positive overall response. Notably, the full extent of these observations was only realized by combination of genomic and transcriptomic information on populations of tumor cells obtained through the laser capture microdissection, a process that was enabled by the use of SIMUL-SEQ. Taken together, our results in this EAC patient both highlight the utility of our method as well as the many benefits of acquiring combined DNA and RNA profiles for genome interpretation and personalized medicine.

Accession Codes

Whole genome sequencing, transcriptome and targeting resequencing data can be found at The National Center for Biotechnology Information (NCBI) Sequence Read Archive (SRA) at the accession number SRP077004, which (as entered by the date of filing of this application) is herein incorporated by reference.

Methods

Sample Acquisition

The male patient-derived fibroblasts used in this study were collected and derived with informed patient consent under a protocol approved by the Institutional Review Board at Stanford University Medical Center (IRB17576). Cells tested negative for mycoplasma and were cultured with DMEM supplemented with 10% FBS. The de-identified male esophageal cancer sample was obtained from Stanford Cancer Institute's Tissue Repository and was exempt from IRB requirements by the Stanford Research Compliance Office. Investigators were not blinded to experimental groups, and no power calculation was performed prior to experiments to ensure detection of a pre-specified effect size.

DNA/RNA Extraction

For the mixing experiments, yeast mRNA was obtained from Clontech (Clontech:636312) and human genomic DNA was isolated using the DNA Mini kit (Qiagen:51304). For all other SIMUL-SEQ experiments, total nucleic acids were extracted using the RNeasy Mini kit (Qiagen:74104) per manufacturer's instructions, except the optional DNase I treatment was not performed. DNA and RNA were then quantified using the Qubit DNA HS and RNA HS (Thermo Fisher: Q32851, Q32852), respectively. For fibroblast experiments, extraction began with 1×10⁶ cells, whereas the LCM tumor library started with approximately 150 μg of tissue (based on isolating ˜150×10⁶ μm³ and assuming an average tissue density of 1.0 g/cm³). The quality of the starting total RNA was measured using Bioanalyzer, with RIN values ranging from 8-10 for LCM-isolated tissue and cells, respectively. For SIMUL-SEQ library preparations, ERCC spike in mixture A (Life Technologies:4456740) was added per manufacturer's instructions prior to the ribosomal RNA depletion step.

Ribosomal Depletion

Ribosomal RNA sequences were depleted from the total nucleic acid mixture using Ribo-Zero gold (Illumina: MRZG126) and following the manufacturer's instructions. To reduce potential hybridization to genomic DNA sequences, however, the standard 70° C. hybridization step was changed to 65° C. Ribosomal RNA depletion began with the recommended amount of total RNA (1-5 μg for LCM-tissue and fibroblasts, respectively). For 50K fibroblast experiments, ˜400 ng of total RNA was used. Following ribosomal RNA depletion, the total nucleic acid mixture was purified using RNA Clean and Concentrator 5 columns (Zymo Research: R1015) and quantified using high sensitivity DNA and RNA Qubit reagents as above.

SIMUL-SEQ Protocol

Unless otherwise noted, reagents were from New England Biosciences (NEB:E7330S) or Illumina (Illumina: FC-121-1031). Simultaneous RNA fragmentation and DNA tagmentation was achieved by mixing 25 μl of TD buffer, 5 μl of TDE, 1 μl RNase III (0.5 U, NEB:E6146S) and 19 μl of DNA/RNA consisting of 30-50 ng of genomic DNA and between 10 and 100 ng of ribo-depleted RNA. This reaction was incubated for 5 minutes at 55° C., and the thermocycler was cooled to 10° C. before the reaction was placed on ice. 100 μl Ampure XP RNAclean beads (Beckman Coulter: A63987), or 2× of the reaction volume, were then added to the reaction and incubated for 10-15 minutes to bind the nucleic acids. The beads were placed on a magnet stand until clear, washed twice with 400 μl of 80% ethanol and dried for 10 minutes at room temperature. The total nucleic acids were eluted from the dried beads using 7 μl of H₂O. To remove secondary RNA structure, 6 μl of the eluate and 1 μl of the 3′ ligation adapter was first heated to 65° C. for 5 minutes and then placed immediately on ice. For ligation of the 3′ adapter to the RNA molecules, 10 μl of 3′ ligation buffer and 3 μl of 3′ ligation enzyme mix were added and incubated for 1 hour at 25° C. in a thermal cycler with the lid heated to 50° C. To reduce adapter-adapter ligation products, 1 μl of the reverse transcription primer (SR RT primer) and 4.5 μl of H₂O were added to the 3′ adapter ligation reaction and incubated in a PCR machine for 5 minutes at 65° C., 15 minutes at 37° C., 15 minutes at 25° C. and held at 4° C. until the next step. To ligate the 5′ adapter, 1 μl of 5′ SR adaptor, which had been previously heated to 70° C. and then placed on ice, along with 1 μl of 5′ ligation buffer and 2.5 μl of 5′ ligase enzyme mix were added to the 3′ adapter ligated and SR RT primer hybridized RNA. This reaction was incubated for 1 hour at 25° C. with the lid heated to 50° C. and then placed on ice. First strand cDNA synthesis was performed by adding 8 μl of first strand reaction buffer, 1 μl of murine RNase inhibitor and 1 μl of ProtoScript II reverse transcriptase to the previous mixture and incubating the reaction for 1 hour at 42° C. with the lid heated to 50° C. 48 μl of Ampure XP beads (Beckman Coulter:A63880), or 1.2× of the reaction volume, were then used to clean up the cDNA and transposed genomic DNA. The beads were incubated for 5-10 minutes with the DNA, washed twice with 80% ethanol and mixed with 26.5 μl of H₂O to elute the DNA. PCR conditions varied depending on if differential PCR was performed. DNA libraries were amplified using standard Nextera indexing primers. RNA libraries were amplified with a custom IS indexing primer AATGATACGGCGACCACCGAGATCTACACTATCCTCTGTTCAGAGTTCTACAG TCCG-s-A (SEQ ID NO:1), where -s- indicates a phosphorothioate bond, and a standard I7 indexing primer. For differential PCR, 25.5 μl of the eluate was combined with 1.25 μl of each RNA indexing primer (10 mM stock) and 12 μl NPM and then thermocycled as follows: 72° C. for 3 minutes, 98° C. for 30 seconds, then 2-7 cycles of 98° C. for 10 seconds, 62° C. for 30 seconds and 72° C. for 3 minutes before a final hold at 4° C. After this hold, the reaction was removed from the thermocycler and combined with 12.5 μl of a master mix comprising 2.5 μl of each DNA indexing PCR primer (5 mM stock), 5 μl of PPC and 5 μl NPM. This combined reaction was then subjected to 5 additional cycles using the same program described above. The fibroblast, LCM and 50K fibroblast SIMUL-SEQ libraries used 2, 4 and 7 cycles of RNA-specific PCR, respectively. The final libraries were cleaned using 66 μl Ampure XP beads as described above and eluted in 12 μl of H₂0. To quality control the dual-indexed libraries, we performed high sensitivity Qubit DNA and Bioanalyzer assays prior to sequencing on Illumina HiSeq or MiSeq paired-end 101 bp reads. A typical SIMUL-SEQ library will be approximately 10 ng/ml, with an average size distribution of ˜350 bp (FIG. 6B). A detailed description of SIMUL-SEQ reagents, equipment and a step by step protocol can be found in Example 2.

Read Processing and Alignment

For both DNA and RNA reads, Cutadapt v1.8.1 (Martin (2011) Cutadapt removes adapter sequences from high-throughput sequencing reads. EMBnet.journal 17:10) was used to trim the paired-end adapter sequences. Only trimmed reads longer than 30 bases and with a quality score>20 were aligned. For the DNA barcoded reads, 5′-CTGTCTCTTATACACATCTCCGAGCCCACGAGAC-3′ (SEQ ID NO:2) and 5′-CTGTCTCTTATACACATCTGACGCTGCCGACGA-3′ (SEQ ID NO:3) sequences were used to trim the adapter sequences. For RNA barcoded reads, 5′-AGATCGGAAGAGCACACGTCTGAACTCCAGTCAC-3′ (SEQ ID NO:4) and 5′-GATCGTCGGACTGTAGAACTCTGAACGTGTAGATC-3 (SEQ ID NO:5) sequences were used to trim the adapter sequences.

DNA libraries were processed and analyzed using the Bina Technologies whole-genome analysis workflow with default settings. Briefly, libraries were mapped with BWA mem 0.7.5 (Li et al. (2009) Bioinformatics 25:1754-1760) to hg19 and then realigned around indels with GATK IndelRealigner (DePristo et al. (2011) Nat. Genet. 43:491-498). Next, base recalibration was performed with GATK BaseRecalibrator taking into account the read group, quality scores, cycle and context covariates. Variants were called with GATK HaplotypeCaller with the parameters—variant_index_type LINEAR—variant_index_parameter 128000. VQSR was used to recalibrate the variants, first with GATK VariantRecalibrator and then ApplyRecalibration. For the cross-contamination analysis shown in FIG. 1B, SIMUL-SEQ DNA-seq indexed reads were mapped to hg19 and SacCer3 using Bowtie2 (Langmead et al. (2012) Nat. Methods 9:357-359) with default settings.

RNA libraries were also processed and analyzed using Bina Technologies RNA analysis using default settings. Briefly, TopHat 2.0.11 (Trapnell et al. (2012) Nat. Protoc. 7:562-78) was used to map libraries to hg19, and Cufflinks (Trapnell et al. (2010) Nat. Biotechnol. 28:511-515) was then used to perform per-sample gene expression analysis. Finally, Cuffdiff was used to find differential expression between replicates and different library types. For cross-contamination analysis shown in FIG. 1B, SIMUL-SEQ RNA-indexed reads were mapped with TopHat to hg19 and SacCer3 using default settings.

DNA and RNA QC Analysis

Coverage plots were calculated from the Bina output. SNV and indel concordance between sequencing libraries was calculated using VCFtools v0.1.12 (Danecek et al. (2011) Bioinformatics 27:2156-2158) on all variants annotated with a “passed” filter. Summary statistics for SNVs were also calculated with VCFtools. Read fractions were calculated with Picard v1.92 (broadinstitute.github.io/picard) for the DNA and RNA sequencing libraries. Strand specificity and gene body coverage were calculated with RSeQC 2.6.2 (Wang et al. (2012) Bioinformatics 28:2184-2185). For the analysis transcripts biotypes, the SIMUL-SEQ RNA data was mapped with TopHat using the Ensembl GENCODE annotations and quantitated with Cufflinks. Genes with FPKM values≧5 were counted. Cuffdiff was used to compare Log 10(FPKM+1) expression values between SIMUL-SEQ RNA libraries and control RNA-seq libraries.

Lorenz Curves

Duplicates were removed from hg19-aligned reads using Picard v1.92, and Bedtools v2.18.0 (Quinlan et al. (2010) Bioinformatics 26:841-842) was used to calculate the coverage at every position in the genome. The file was then sorted by coverage, and cumulative sums for the fraction of the covered genome and the fraction of total mapped bases were calculated using custom scripts.

ERCC Analysis

TopHat was used to align reads to ERCC reference using default settings. Next, duplicate reads were removed using Picard MarkDuplicates, and FeatureCounts (Liao et al. (2014) Bioinformatics 30:923-930) was used to determine the total read counts for each ERRC transcript. Read counts were then normalized across transcripts and libraries using the RPKM methodology (i.e., reads/kb of transcript/million mapped reads). ERCC RPKM measurements for SIMUL-SEQ and RNA-seq replicates were averaged, zero values were set to 1 and then Log 10-transformed.

Droplet Digital PCR (ddPCR)

DNA:RNA ratios of between 5-10:1 are optimal for whole genome and whole transcriptome sequencing of human samples. ddPCR experiments were performed according to manufacturer's guidelines (Droplet Digital PCR Application Guide, Bulletin 6407 Rev A) using a Bio-Rad QX200 system. Briefly, custom qPCR assays were designed to the unique the DNA-seq and RNA-seq library adapter sequences and purchased from IDT as PrimeTime Std qPCR Assays. These assays incorporated HPLC-purified probes with 5′ HEX or 6-FAM fluorophores and internal ZEN and 3′ Iowa Black FQ dual quenchers. Twenty microliter ddPCR reactions were assembled using diluted SIMUL-SEQ libraries (2 μl of a 10⁻⁶ dilution was typically sufficient but will vary depending on the starting library concentration). The ddPCR reactions were then subjected to the following cycling program: 10 minutes at 95° C., 40 cycles of 30 seconds at 95° C. and 1 minute at 60° C., 10 minutes at 98° C. and a hold at 4° C. Triplicate reactions were done for each sample, and quantitation was performed using QuantaSoft version 1.3.2.

Laser Capture Microdissection (LCM)

For LCM, 7 μm cryosections were placed onto 76×26 PEN glass slides (Leica: #11505158) and stored at −80° C. for up to 4 days. To guide the isolation process, serial sections were immunofluorescently stained with Keratin 8 (1:100; Abcam:ab668-100) and counterstained with Hoechst 33342 dye (2 mg/ml in PBS), marking the tumor epithelium and nuclei, respectively. On the day of laser capture, the LCM slides were stained with Cresyl violet according to the manufacturer's protocol (LCM staining kit, Ambion: #AM1935). Immediately following staining, a Leica AS LIVID system was used to isolate ˜150×10⁶ μm³ (or ˜150 μg) of esophageal adenocarcinoma tumor tissue. The LCM-isolated tissue was then subjected to the SIMUL-SEQ protocol and 727,341,682 DNA and 191,398,961 RNA 101 bp paired-end reads were obtained using an IIlumina HiSeq2000 machine. For all transcriptome analyses using SIMUL-SEQ RNA tumor data, 116,217,162 reads were analyzed.

Somatic Variant Analysis

Somatic variant analysis was performed using Bina tumor-normal whole-genome calling workflow. Briefly, somatic variants with a Bina ONCOSCORE of greater than or equal to 5 were considered high confidence and reported. To identify somatic variants and generate the ONCOSCORE, Bina integrates JointSNVMix 0.7.5 (Roth et al. (2012) Bioinformatics 28:907-913), Mutect 2014.3-24-g7dfb931 (Cibulskis et al. (2013) Nat. Biotechnol. 31:213-219), Somatic Indel Detector 2014.3-24-g7dfb931, Somatic Sniper 1.0.4 (Larson et al. (2012) Bioinformatics 28:311-317) and Varscan 2.3.7 (Koboldt et al. (2012) Genome Res. 22:568-576) outputs. GATK ASEReadCounter was used to determine the variant and reference expression counts for somatic SNV positions in the tumor transcriptome data.

To determine large somatic structural variants (SVs), CREST (Wang et al. (2011) Nat. Methods 8:652-654) was run on the tumor-normal paired genomic data. To refine the variant calls, we only reported SVs with greater than 5 supporting reads on both the 3 prime and 5 prime arms of the variant, which resulted in 142 total potential genomic SVs. Somatic SVs resulting in expressed gene fusions, were independently determined using the INTEGRATE software package (Zhang et al. (2016) Genome Res. 26(1):108-118), which incorporates tumor RNA sequencing data along with paired tumor-normal genome sequencing data. To refine this expressed fusion list, we only reported fusions with no evidence in the normal DNA data and at least one read of evidence for both the tumor DNA and RNA, which resulted in 9 potential expressed gene fusions. Circos software 0.63 (Krzywinski et al. (2009) Genome Res. 19:1639-1645) was used to display somatic variation.

Loss of Heterozygosity (LOH)

For the loss of heterozygosity analysis, heterozygous positions in the normal were selected in the VCF file using SNPsift (Cingolani et al. (2012) Front. Genet. 3:1-9). GATK SelectVariants was then used to interrogate these heterozygous positions in the tumor VCF, classifying them as heterozygous or homozygous alternative. Heterozygous positions in the normal that were not present in the tumor VCF were considered homozygous reference and counted as LOH positions.

Allele-Specific Expression (ASE)

To examine LOH at the level of gene expression, ASE in the tumor RNA was calculated for heterozygous positions called in the normal using ASEQ (Romanel et al. (2015) BMC Med. Genomics 8:9). Briefly, GENOTYPE mode was run on a bam file derived from the paired normal genome, with the following options: mbq=20 mrq=1 mdc=5 htperc=0.2. Next, ASE mode was run using a bam file from the tumor RNA, with the following options: mbq=20 mrq=20 mdc=10 pht=0.01 pft=0.01. This analysis was performed using an hg19 Ensembl transcript model and identified 21,797 transcripts—corresponding to 6,698 independent gene symbols—as exhibiting ASE. Circos was used to display the number of ASE transcripts in 100 kb bins.

Targeted Resequencing of KIF3B Locus

Overlapping primer sets were designed to capture all of the coding exons of the KIF3B locus. Genomic DNA was isolated from 50 formalin-fixed paraffin embedded (FFPE) tumor samples as well as 26 paired normal samples using an AllPrep DNA/RNA FFPE kit (Qiagen, 80204), according to manufacturer's instruction. The original sample (02-28923-C9) that was subjected to the SIMUL-SEQ protocol was included as a positive control. The gDNA concentrations were normalized to 50 ng/μl and subjected to amplification on a Fluidigm Axess Array system, following manufacturer's recommendation (FC1 Cycler v1.0 User Guide rev A4). The resultant libraries were pooled, sequenced on a single HiSeq2000 lane and mapped using bowtie. SAMtools (Li et al. (2009) Bioinformatics 25:2078-2079) was used to generate a pileup, and SNVs were identified using four criteria: mapped to a targeted region, allele read fraction of >10%, mapping quality of >10 and coverage of >500. Using these criteria, three variants in KIF3B were identified and subsequently validated using pyrophosphate sequencing. A single tumor-normal pair (00-18224-A2) displayed a substantially higher number of variant calls yet a lower number of uniquely mapped reads, suggesting that these samples harbored increased rates of PCR errors induced by low quality genomic DNA. Therefore, variants identified in these samples were not reported.

Kinesin-Microtubule Interaction Assays

Full-length kinesin proteins exhibit poor solubility in bacteria (Stock et al. (2001) Methods Mol. Biol. 164, 43-48). Therefore, wild-type and R293W mutant motor domains (amino acids 1-365) were amplified using the following primers: CATATGTCAAAGTTGAAAAGCTCAG (SEQ ID NO:6) and CTCGAGCTAGAGCCGAGCAATCTCTTCCT (SEQ ID NO:7). The PCR products were digested with NdeI/XhoI restriction enzymes and cloned into NdeI/XhoI digested pET28a backbone, tagging the KIF3B motor domains on the N-terminus. Recombinant KIF3B was purified using nickel affinity purification (FIGS. 12A, 12B). Briefly, bacterial pellets were lysed for 30 minutes on ice in lysis buffer (50 mM PIPES, pH 8.0, 1 mM MgCl₂, 250 mM NaCl₂, 250 μg/ml lysozyme, 250 mM ATP and protease inhibitors (Roche; 04693132001)). Lysates were pulse sonicated for 3 cycles of 18% amplitude (Bronson) for 5 seconds (0.5 seconds on/1 second off) followed by 1 minute on ice. Lysates were then cleared by centrifugation for 10 minutes at 4° C. and maximum speed. Cleared lysates were incubated with His-tag magnetic beads (Life Technologies, 10103D) for 1 hour at 4° C., washed 2× in washing buffer (50 mM PIPES, pH 8.0, 1 mM MgCl₂, 250 mM NaCl₂, 50 mM Imidazole) supplemented with 250 mM ATP followed by an additional 2 washes in buffer excluding ATP. Beads were subsequently eluted in 25 mM PIPES, pH 8.0, 2 mM MgCl₂, 125 mM NaCl₂, and 250 mM Imidazole. Kinesin ATPase end-point biochemical assays (Cytoskeleton, BK053) were performed in duplicate according to manufacturer's instructions with 0.4 μg of recombinant protein and increasing amounts of polymerized microtubules (see, FIG. 5B).

TABLE 1
Selected expressed somatic nonsynonymous
variants in cancer-related genes

| | DNA | RNA counts | | Cosmic |
|---|---|---|---|---|
| Gene | [Ref/Alt] | [Ref/Alt] | Protein | census |
| TP53 | T/C | 0/76 | Y220C | yes |
| ATM | C/T | 102/37 | R45W | yes |
| EWSR1 | C/T | 26/9 | P122L | yes |
| KIF3B | C/T | 170/64 | R293W | no |
| MCM3AP | G/A | 5/127 | R1207C | no |
| FAT1 | C/T | 11/44 | V1274I | no |
| MADD | G/A | 59/19 | R225Q | no |
| LRP1 | G/T | 16/3 | D2106Y | no |
| H2AFY | G/A | 13/43 | R4C | no |
| ZNF615 | T/C | 0/10 | N154S | no |
| CSTF1 | G/A | 51/68 | G26S | no |

TABLE 2
Simul-seq and control library read counts and mapping rates.

| | DNA | RNA | DNA | RNA |
|---|---|---|---|---|
| Experiment | Read Count | Read Count | Mapping | Mapping |
| Yeast/Human | 2,545,087 | 1,976,515 | 0.975 | 0.880 |
| rep1 | | | | |
| Yeast/Human | 2,328,071 | 1,728,676 | 0.975 | 0.889 |
| rep2 | | | | |
| Simul-seq rep1 | 560,218,621 | 57,091,162 | 0.934 | 0.786 |
| Simul-seq rep2 | 376,930,142 | 67,062,122 | 0.914 | 0.736 |
| RNA-seq rep1 | N/A | 40,463,932 | N/A | 0.807 |
| RNA-seq rep2 | N/A | 39,767,156 | N/A | 0.715 |
| DNA-seq rep1 | 558,622,492 | N/A | 0.909 | N/A |
| Simul-seq EAC | 727,341,682 | 191,398,961 | 0.951 | 0.794 |
| DNA-seq | 524,195,632 | N/A | 0.952 | N/A |
| normal esophagus | | | | |
| Simul-seq 50K | 484,665,205 | 54,002,313 | 0.932 | 0.877 |
| rep1 | | | | |
| Simul-seq 50K | 482,405,461 | 74,730,465 | 0.933 | 0.807 |
| rep2 | | | | |

### EXAMPLE 2

### SIMUL-SEQ Reagents, Equipment and Protocol

### Detailed Description of SIMUL-SEQ Reagents, Equipment and Protocol

Reagents
0.2 mL PCR tubes
Molecular Probes Qubit assay tubes: Q32856
1.5 mL Eppendorf DNA LoBind microcentrifuge tubes: 022431021
Qiagen RNeasy mini: 74104
Qubit dsDNA HS kit: Q32851
Qubit RNA HS kit: Q32852
Illumina RiboZero Magnetic Gold: MRZG126
Zymo Research RNA Clean and Concentrator 5 kit: R1015
NEB Small RNA Library Preparation Kit: E7330S
Illumina Nextera Library Preparation Kit: FC-121-1031
Illumina Nextera Index Kit: FC-121-1012
NEB RNase III: E6146S
Beckman Coulter Ampure XP RNAclean beads: A63987
Beckman Coulter Ampure XP beads: A63880
HPLC-purified custom NEB PCR primers (*denotes phosphorothioate bond) (all 100 nmole from
IDT):

NEB SR 503
(SEQ ID NO: 8)
5′-AATGATACGGCGACCACCGAGATCTACACTATCCTCTGTTCAGAG
TTCTACAGTCCG*A-3′
NEB SR 505
(SEQ ID NO: 9)
5′-AATGATACGGCGACCACCGAGATCTACACGTAAGGAGGTTCAGAGT
TCTACAGTCCG*A-3′
NEB 702
(SEQ ID NO: 10)
5′-CAAGCAGAAGACGGCATACGAGATCTAGTACGGTGACTGGAGTTC
AGACGTGTGCTCTTCCGATC*T-3′
NEB 703
(SEQ ID NO: 11)
5′-CAAGCAGAAGACGGCATACGAGATTTCTGCCTGTGACTGGAGTTCA
GACGTGTGCTCTTCCGATC*T-3′

(Optional Reagents)
IDT PrimeTime Std qPCR Assays for custom ddPCR-based library quantification:

| DNA forward primer (9.0 nM): | |
|---|---|
| 5′-AAGCAGAAGACGGCATACGAG-3′ | (SEQ ID NO: 12) |
| DNA reverse primer (9.0 nM): | |
| 5′-GCGACCACCGAGATCTACAC-3′ | (SEQ ID NO: 13) |

DNA 5′ probe (2.5 nM; HPLC-purified):
5′-CTTATACACATCTGACGCTGCCGACG-3′ (SEQ ID NO:14) with 5′-6-FAM fluorophore and internal ZEN and 3′-Iowa Black FQ dual quenchers
DNA 3′ probe (2.5 nM; HPLC-purified):
5′-TCTCTTATACACATCTCCGAGCCCACG-3′ (SEQ ID NO:15) with 5′-HEX fluorophore and internal ZEN and 3′-Iowa Black FQ dual quenchers
RNA forward primer (9.0 nM): 5′-AAGAGAAGACGGCATACGAG-3′ (SEQ ID NO:16)
RNA reverse primer (9.0 nM): 5′-GCGACCACCGAGATCTACAC-3′ (SEQ ID NO:17)
RNA 5′ probe (2.5 nM; HPLC-purified):
5′-CTGTTCAGAGTTCTACAGTCCGACGATC-3′ (SEQ ID NO:18) with 5′-6-FAM fluorophore and internal ZEN and 3′-Iowa Black FQ dual quenchers
RNA 3′ probe (2.5 nM; HPLC-purified):
5′-ACTGGAGTTCAGACGTGTGCTCTTCC-3′ (SEQ ID NO:19) with 5′-HEX fluorophore and internal ZEN and 3′-Iowa Black FQ dual quenchers
Bio-Rad 2× digital droplet PCR supermix for probes (No dUTP): 186-3023 Bio-Rad droplet generation oil for probes: 1863005
Bio-Rad DG8 Cartridges: 1864008
Bio-Rad DG8 Gaskets: 1863009
Bio-Rad ddPCR Droplet Reader Oil: 1863004
Eppendorf 96-well PCR plate: 951020362

### Equipment

Microcentrifuge (general lab supplier)
Qubit Fluorometer (Life Technologies)
Magnetic stand for 1.5 ml tubes (general lab supplier)
Thermal cycler with programmable lid temperature (general lab supplier)
Heat block (general lab supplier)
QX200 ddPCR system, with PX1 PCR Plate Sealer (Bio-Rad) (optional equipment)

### Procedure

### A) Nucleic Acid Isolation:

1. Extract total nucleic acids from cell or tissue samples using the Qiagen RNeasy mini kit per manufacturer's instructions, except without the optional DNase I treatment. Elute total nucleic acids in 30 μl of molecular biology grade, nuclease free H₂0 (henceforth called H₂0).

2. Use dsDNA Qubit to quantify genomic DNA concentration and RNA Qubit to quantify total RNA concentration for subsequent RiboZero depletion.

### B) RiboZero Magnetic Gold Ribosomal RNA Depletion:

1. Starting with between 0.5-5 μg of total RNA as measured with the RNA Qubit (with concomitant genomic DNA present), use the Illumina RiboZero Magnetic Gold Depletion kit per manufacturer's instructions with the following change, for step 2.3 change temperature of incubation to 65° C. from 68° C.

2. Use the Zymo Research RNA clean and concentrator 5 cleanup kit following the manufacturer's protocol to recover all nucleic acids>17 nucleotides and elute in 12 μl of H₂0.

3. Use dsDNA Qubit to quantify genomic DNA (gDNA) concentration and RNA Qubit to quantify RNA concentration for tagmentation/fragmentation reaction.

Protocol can be safely paused overnight here if necessary. Store samples at −80° C.

### C) Simultaneous Tagmentation of Genomic DNA and Fragmentation of RNA:

1. Set up the following reaction with 50 ng of genomic DNA and the corresponding amount of ribosomally depleted RNA in a 0.2 mL PCR tube. Between 5-100 ng of starting RNA should be present per 50 ng of gDNA.
a. X μl total nucleic acids (50 ng gDNA)
b. 25 μl Tagment DNA (TD) buffer (Nextera kit)
c. 5 μl Tagment DNA enzyme (TDE) (Nextera kit)
d. Y μl H₂0 (50 μl total reaction volume)
e. 1 μl RNase III (1:1 dilution in TD buffer; 0.5 units of RNase III)

2. Mix well (50 μl) and use a thermal cycler to incubate at 55° C. for 5 minutes and cool to 10° C.; immediately place on ice and proceed to next step.

### D) Ampure Cleanup of Tagmentation/Fragmentation Reaction:

1. Transfer sample to 1.5 mL Eppendorf tube and add 100 μl (or 2× of reaction volume) of Ampure XP RNA clean beads to the 50 μl reaction.

2. Incubate for 10-15 minutes at room temperature and then place on magnetic stand.

3. Wash 2× with 400 μl of 80% ethanol.

4. Air dry for 10 minutes and resuspend in 7 μl of H₂0.

### E) Removal of RNA Secondary Structure:

1. Transfer 6 μl of the elution from the tube on the magnetic stand to a new 0.2 mL PCR tube.

2. Add 1 μl of 3′ adapter (NEB small RNA kit).

3. Incubate in preheated thermal cycler for 2 minutes at 65° C. and immediately place on ice.

### F) 3′ Adapter Ligation:

1. Set up the following reaction on ice to ligate the 3′ adapter onto the RNA, using reagents from the NEB small RNA library preparation kit.
a. 10 μl 3′ Ligation Buffer (2×)
b. 3 μl 3′ Ligation Enzyme Mix

2. On ice add 13 μl of 3′ ligation master mix to the 7 μl of denatured gDNA/RNA.

3. Mix well (20 μl) and incubate for 1 hour at 25° C. in thermal cycler, with lid heated to 55° C.; hold at 4° C.

### G) Hybridization of Reverse Transcription Oligo:

1. Set up the following reaction on ice to hybridize the RT oligo to the 3′ adapter, using reagents from the NEB small RNA library preparation kit.
a. 4.5 μl H₂0
b. 1 μl SR RT Primer

2. Add 5.5 μl of diluted RT primer to the 20 μl 3′ ligation reaction.

3. Mix well (25.5 μl) and use a thermal cycler to incubate the samples for 5 minutes at 65° C., 15 minutes at 37° C. and then 15 minutes at 25° C.; hold at 4° C.

4. With ˜5 minutes remaining, prepare the 5′ SR adaptor by resuspending in 120 μl of H₂0.

5. Aliquot 1.1× of resuspended 5′ SR adaptor and denature for 2 minutes at 70° C.; immediately place on ice. Store excess adapter in aliquots at −80° C. for subsequent use.

### H) Ligate 5′ SR Adaptor:

1. Set up the following reaction on ice to ligate the 5′ adaptor onto the RNA, using reagents from the NEB small RNA library preparation kit.
a. 1 μl 5′ SR adaptor
b. 1 μl 5′ Ligation Reaction Buffer
c. 2.5 μl 5 Ligase Enzyme Mix

2. Add 4.5 μl of the 5′ ligation master mix to the 25.5 μl RT hybridization reaction.

3. Mix well (30 μl) and incubate for 1 hour at 25° C., with lid heated to 55° C.; hold at 4° C.

I) cDNA Synthesis:

1. Set up the following reaction on ice to make first strand cDNA master mix, using reagents from the NEB small RNA library preparation kit.
a. 8 μl First Strand Reaction Buffer (5×)
b. 1 μl Murine RNase Inhibitor
c. 1 μl ProtoScript II Reverse Transcriptase

2. Transfer 30 μl of 5′ ligation reaction to a new 0.2 mL PCR tube and add 10 μl of cDNA synthesis master mix.

3. Mix well (40 μl) and use a thermal cycler to incubate for 1 hour at 50° C., with lid heated to 55° C.; hold at 4° C.

J) Ampure Cleanup of gDNA and cDNA Synthesis Reaction:

1. Transfer sample to new 1.5 mL Eppendorf and add 1.2× Ampure XP beads.

2. Incubate for 5-10 minutes at room temperature; place on magnet.

3. Wash 2× with 400 μl of 80% ethanol.

4. Air dry for 5-10 minutes and resuspend in 26.5 μl of H₂0; place on magnet.

5. Transfer 25.5 μl of elution to a new 0.2 mL PCR tube.

Protocol can be safely paused overnight here if necessary. Store sample at −20° C.

K) gDNA/cDNA Library PCR Amplification:

1. Set up the following PCR reaction to enrich for the cDNA, using PCR reagents from the Nextera library preparation kit and custom multiplex primers (NEB 70× and NEB SR 50× for forward and reverse, respectively). More guidelines for cycle number can be found in the Critical Steps section below.
a. 25.5 μl gDNA/cDNA library mixture
b. 1.25 μl custom NEB SR 50× (10 μM stock)
c. 1.25 μl custom NEB 70× (10 μM stock)
d. 12 μl NPM

2. Mix well (40 μl) and perform thermocycling as follows for 2-7 cycles, depending on RNA input.
a. 72° C., 3 minutes
b. 98° C., 30 seconds
Repeat following cycle 2-7 times
c. 98° C., 10 seconds
d. 62° C., 30 seconds
e. 72° C., 3 minutes
f. Hold at 4° C.

3. Once the PCR reaction is at 4° C., add the PCR master mix below to amplify the final gDNA/cDNA libraries, using PCR reagents and primers from the Nextera library preparation kit.
a. 40 μl gDNA/cDNA library
b. 2.5 μl N70× (5 μM stock)
c. 2.5 μl N50× (5 μM stock)
d. 5 μl PPC
e. 5 μl NPM

4. Mix well (55 μl) and perform thermocycling as follows for 5 cycles
a. 72° C., 3 minutes
b. 98° C., 30 seconds

5. Repeat following cycle 5 times
c. 98° C., 10 seconds
d. 62° C., 30 seconds
e. 72° C., 3 minutes
f. Hold at 4° C.

### L) Ampure Cleanup of Final Library:

1. Transfer sample to new 1.5 mL Eppendorf and add 1.2× Ampure XP beads to 55 μl reaction.

2. Incubate for 5-10 minutes at room temperature and place on magnet.

3. Wash 2× with 400 μl of 80% ethanol.

4. Air dry for 5-10 minutes and resuspend in 12.5 μl of H₂0; place on magnet.

5. Transfer 12 μl of elution to a new 1.5 mL Eppendorf tube.

6. Use Qubit dsDNA HS to quantify final library concentration. Agilent Bioanlyzer High Sensitivity kit can be used to visualize the library size. A typical SIMUL-SEQ library will be approximately 10 ng/μl, with an average size distribution of ˜350 bp (FIG. 1B).

M) (Optional) ddPCR Quantification of Libraries

1. ddPCR experiments are performed according to manufacturer's guidelines, using a Bio-Rad QX200 system.

2. Equally mix 5′ and 3′ IDT PrimeTime Std qPCR assays for ddPCR-based library quantification of either the RNA (NEB) or the DNA (Nextera) constituents of the SIMUL-SEQ library. Note, assaying both ends ensures that both 5′ and 3′ adapters are correct.

3. Assemble triplicate 20 μl ddPCR reactions at room temperature:
a. 10 μl of ddPCR 2× super mix (Bio-Rad)
b. 1 μl of combined NEB or Nextera ddPCR assay mixes
c. 2 μl of diluted SIMUL-SEQ libraries (10⁻⁶ dilution often is sufficient but will vary depending on the starting library concentration).
d. 7 μl of H₂0

4. Generate droplets by adding the 20 μl ddPCR reactions to the sample wells of a DG8 cartridge and then adding 70 μl of droplet generation oil for probes to oil wells; place a DG8 gasket over the cartridge and insert into the QX200 droplet generator. Transfer droplets into an Eppendorf 96-well PCR plate and repeat until droplets have been produced for all of the ddPCR reactions.

5. Cover PCR plate with foil using the plate sealer and subject the ddPCR reactions to the following PCR cycling program:
a. 10 minutes at 95° C.

40 cycles of:
b. 30 seconds 95° C.
c. 1 minute at 60° C.
d. Followed by 10 minutes at 98° C.
e. Hold at 4° C.

6. Transfer plate to the QX200 droplet reader and quantitate library ratios with QuantaSoft software (FIGS. 2A, 2B).

Critical Steps

Use proper RNA handling procedures throughout as RNases can lead to degradation and lower quality libraries.

Step B-3: Measurement of the gDNA prior to tagmentation is critical because 50 ng should be added to the reaction for optimal results.

Step C-1: Tagmentation/fragmentation step needs to be carried out without interruption as prolonged RNase III activity may over fragment the RNA.

Step D-1: Use of Ampure XP RNA beads is recommended, as initial experiments with column based clean ups exhibited increased bias in Nextera DNA-seq data.

Step D-2: Full incubation time for the Ampure XP RNA cleanup is critical, as RNA does not bind as efficiently to the beads as DNA.

Step K-1: Choosing the correct number of cycles for the cDNA enrichment PCR is critical for a balanced ratio of DNA/RNA reads in the final library (FIG. 1c). General guidelines based on the starting amount of ribosomally depleted RNA are as follows: 7 cycles for 5-15 ng, 5 cycles for 15-30 ng, 3 cycles for 30-50 ng, 2 cycles for >50 ng. Variability between sources of starting material may affect final library ratios. It is recommended that the user tests the number of PCR cycles when changing a source of starting material.

Step K-2: For the final library amplification PCR, 5 cycles worked well for all experiments that were undertaken.

Step M: DNA:RNA ratios of between 5-10:1 are optimal for whole genome and whole transcriptome sequencing of human samples. ddPCR ratios are well correlated to final read counts (FIG. 1D) if alternative ratios are desired.

Sequencing must be performed with Illumina dual index reads to ensure proper adapter pairing. NEB PCR primers have been remade with normal Nextera indices for ease of pooling (see reagents). Nextera low plex pooling guidelines should be followed to balance index reads, allowing for optimum library yields. Finally, this library design is only compatible with MiSeq and HiSeq Illumina platforms.

Although preferred embodiments of the subject invention have been described in some detail, it is understood that obvious variations can be made without departing from the spirit and the scope of the invention as defined herein.

What is claimed is:

1. A method of sequencing RNA and DNA, the method comprising:
a) providing a biological sample comprising nucleic acids;
b) isolating the nucleic acids from the sample;
c) fragmenting the DNA by contacting the nucleic acids with a Class 2 transposase loaded with a transposable oligonucleotide adapter comprising a common priming site for DNA-specific amplification, wherein the transposase catalyzes cleavage of the DNA into a plurality of DNA fragments and ligation of the oligonucleotide adapter to each DNA fragment at the 5′ ends of its DNA strands;
d) fragmenting the RNA by contacting the nucleic acids with RNase III, such that the RNase III cleaves the RNA into a plurality of RNA fragments;
e) ligating a 3′ oligonucleotide adapter comprising a 3′ common priming site for RNA-specific amplification to the 3′ end of each RNA fragment using an RNA ligase;
f) ligating a 5′ oligonucleotide adapter comprising a 5′ common priming site for RNA-specific amplification to the 5′ end of each RNA fragment using an RNA ligase;
g) adding reverse transcriptase such that a plurality of cDNA fragments is produced from the plurality of RNA fragments;
h) amplifying the plurality of DNA fragments with a set of DNA indexing primers that selectively bind to the common priming sites for DNA-specific amplification to produce a plurality of DNA amplicons, wherein each amplicon comprises a common priming site for DNA-specific sequencing and a DNA index sequence;
i) amplifying the plurality of cDNA fragments with a set of RNA indexing primers that selectively bind to the common priming sites for RNA-specific amplification to produce a plurality of cDNA amplicons, wherein each amplicon comprises a common priming site for RNA-specific sequencing and an RNA index sequence; and
j) sequencing the DNA amplicons and the cDNA amplicons, wherein the DNA index sequence is used to identify DNA sequences and the RNA index sequence is used to identify RNA sequences.
wherein (b)-(j) are performed with the RNA and DNA nucleic acids pooled together.

2. The method of claim 1, wherein said isolating the nucleic acids comprises lysing a cell in the biological sample and extracting the nucleic acids from the cell.

3. The method of claim 1, further comprising removing ribosomal RNA from the nucleic acids prior to said fragmenting the DNA and RNA.

4. The method of claim 1, further comprising purifying the RNA or DNA prior to sequencing.

5. The method of claim 1, wherein said amplifying the RNA or DNA comprises performing digital droplet PCR.

6. The method of claim 1, wherein said amplifying the RNA or DNA comprises performing clonal amplification.

7. The method of claim 6, wherein the clonal amplification comprises emulsion PCR or bridge amplification.

8. The method of claim 1, wherein said amplifying the plurality of DNA fragments with the set of DNA indexing primers is performed with different numbers of cycles of PCR than said amplifying the plurality of cDNA fragments with the set of RNA indexing primers.

9. The method of claim 1, wherein the transposase is hyperactive.

10. The method of claim 1, wherein the transposase is a Tn5 transposase.

11. The method of claim 1, wherein the DNA and the RNA are from a single cell or a selected population of cells of interest.

12. The method of claim 11, wherein the cell is a eukaryotic cell or a prokaryotic cell.

13. The method of claim 1, wherein the biological sample is from an animal, plant, bacterium, fungus, protist, archaeon, or virus.

14. The method of claim 1, wherein the biological sample comprises a genetically aberrant cell, cancer cell, or rare blood cell.

15. The method of claim 11, wherein the cell is a human cell.

16. The method of claim 11, wherein the cell is a live cell or a fixed cell.

17. The method of claim 1, wherein the DNA and the RNA are from a sample of micro-dissected tissue.

18. The method of claim 1, wherein the DNA and the RNA are from a biopsy.

19. The method of claim 1, wherein said sequencing comprises paired-end sequencing or single-read sequencing.

20. The method of claim 1, wherein said sequencing comprises next-generation sequencing (NGS).

21. The method of claim 1, further comprising assembling the DNA sequences or the cDNA sequences to produce longer sequences or complete sequences of genes or RNA transcripts.

22. The method of claim 1, further comprising identifying a mutation in the DNA or RNA.

23. The method of claim 22, wherein the mutation is an insertion, a deletion, or a substitution.

24. The method of claim 22, wherein the mutation is a single nucleotide variation.

25. The method of claim 22, wherein the mutation is associated with a phenotype of interest.

26. The method of claim 1, further comprising detecting genomic copy number variation.

27. The method of claim 1, further comprising performing transcriptome quantification or isoform analysis.

28. The method of claim 1, wherein at least one RNA indexing PCR primer comprises a nucleotide sequence of SEQ ID NO:1 or a nucleotide sequence having at least 95% identity to the nucleotide sequence of SEQ ID NO:1.

29. The method of claim 1, wherein (a)-(j) are carried out in one container.

30. A kit for performing the method of claim 1 comprising:
a) a Class 2 transposase;
b) a transposable oligonucleotide comprising an oligonucleotide adapter comprising a common priming site for DNA-specific amplification;
c) a 5′ oligonucleotide adapter comprising a 5′ common priming site for RNA-specific amplification;
d) a 3′ oligonucleotide adapter comprising a 3′ common priming site for RNA-specific amplification;
k) an RNase III;
l) an RNA ligase;
m) a reverse transcriptase;
n) a DNA polymerase;
o) a set of DNA indexing PCR primers; and
p) a set of RNA indexing PCR primers.

31. The kit of claim 30, further comprising reagents for performing next-generation sequencing.

32. The kit of claim 30, wherein the set of RNA indexing PCR primers comprises a primer comprising a nucleotide sequence of SEQ ID NO:1 or a nucleotide sequence having at least 95% identity to the nucleotide sequence of SEQ ID NO:1.

33. An oligonucleotide comprising a nucleotide sequence of SEQ ID NO:1 or a nucleotide sequence having at least 95% identity to the nucleotide sequence of SEQ ID NO:1.


SEQUENCE LISTING (19 sequences)

SEQ ID NO: 1 - DNA, 58 bp, Artificial Sequence
  I5 indexing primer
  source (1)..(58): sequence is synthesized
aatgatacggcgaccaccgagatctacactatcctctgttcagagttctacagtccga

SEQ ID NO: 2 - DNA, 34 bp, Artificial Sequence
  DNA adapter sequence
  source (1)..(34): sequence is synthesized
ctgtctcttatacacatctccgagcccacgagac

SEQ ID NO: 3 - DNA, 33 bp, Artificial Sequence
  DNA adapter sequence
  source (1)..(33): sequence is synthesized
ctgtctcttatacacatctgacgctgccgacga

SEQ ID NO: 4 - DNA, 34 bp, Artificial Sequence
  RNA adapter sequence
  source (1)..(34): sequence is synthesized
agatcggaagagcacacgtctgaactccagtcac

SEQ ID NO: 5 - DNA, 35 bp, Artificial Sequence
  RNA adapter sequence
  source (1)..(35): sequence is synthesized
gatcgtcggactgtagaactctgaacgtgtagatc

SEQ ID NO: 6 - DNA, 25 bp, Artificial Sequence
  mutant motor domain primer
  source (1)..(25): sequence is synthesized
catatgtcaaagttgaaaagctcag

SEQ ID NO: 7 - DNA, 29 bp, Artificial Sequence
  mutant motor domain primer
  source (1)..(29): sequence is synthesized
ctcgagctagagccgagcaatctcttcct

SEQ ID NO: 8 - DNA, 58 bp, Artificial Sequence; identical to SEQ ID NO: 1
  NEB SR 503 primer
  source (1)..(58): sequence is synthesized

SEQ ID NO: 9 - DNA, 58 bp, Artificial Sequence
  NEB SR 505 primer
  source (1)..(58): sequence is synthesized
aatgatacggcgaccaccgagatctacacgtaaggaggttcagagttctacagtccga

SEQ ID NO: 10 - DNA, 66 bp, Artificial Sequence
  NEB 702 primer
  source (1)..(66): sequence is synthesized
caagcagaagacggcatacgagatctagtacggtgactggagttcagacgtgtgctcttccgatct

SEQ ID NO: 11 - DNA, 66 bp, Artificial Sequence
  NEB 703 primer
  source (1)..(66): sequence is synthesized
caagcagaagacggcatacgagatttctgcctgtgactggagttcagacgtgtgctcttccgatct

SEQ ID NO: 12 - DNA, 21 bp, Artificial Sequence
  IDT DNA forward primer
  source (1)..(21): sequence is synthesized
aagcagaagacggcatacgag

SEQ ID NO: 13 - DNA, 20 bp, Artificial Sequence
  IDT DNA reverse primer
  source (1)..(20): sequence is synthesized
gcgaccaccgagatctacac

SEQ ID NO: 14 - DNA, 26 bp, Artificial Sequence
  IDT DNA 5′ probe
  source (1)..(26): sequence is synthesized
cttatacacatctgacgctgccgacg

SEQ ID NO: 15 - DNA, 27 bp, Artificial Sequence
  IDT DNA 3′ probe
  source (1)..(27): sequence is synthesized
tctcttatacacatctccgagcccacg

SEQ ID NO: 16 - DNA, 20 bp, Artificial Sequence
  IDT RNA forward primer
  source (1)..(20): sequence is synthesized
aagagaagacggcatacgag

SEQ ID NO: 17 - DNA, 20 bp, Artificial Sequence
  IDT RNA reverse primer
  source (1)..(20): sequence is synthesized
gcgaccaccgagatctacac

SEQ ID NO: 18 - DNA, 28 bp, Artificial Sequence
  IDT RNA 5′ probe
  source (1)..(28): sequence is synthesized
ctgttcagagttctacagtccgacgatc

SEQ ID NO: 19 - DNA, 26 bp, Artificial Sequence
  IDT RNA 3′ probe
  source (1)..(26): sequence is synthesized
actggagttcagacgtgtgctcttcc